SEQUENCE LISTING (17 sequences)

SEQ ID NO: 1 - protein, 21 aa, Homo sapiens
  VARIANT 18: Xaa = Arg or Ser
VQLQESGPGLLKPSETLXLTN

SEQ ID NO: 2 - protein, 19 aa, Homo sapiens
DIQMTQSPSSLSASVGDRV

SEQ ID NO: 3 - protein, 106 aa, Homo sapiens
DIQMTQSPSSLSASVGDRVTITCRASQSISSYLNWYQQKPGKAPKVLIYAASLRSGVPSRFSGSGSGTDFTLTVSSLQPEDFATYYCQQSYHTPWTFGQGTKVEIK

SEQ ID NO: 4 - protein, 107 aa, Homo sapiens [sequence omitted]

SEQ ID NO: 5 - protein, 118 aa, Homo sapiens
QVQLQESGPGLLKPSETLSLTCTVSGGSVSLYYWSWIRQSPGKEPEWIGYIYSSGSTDYNPSLRSRVTISLDTSNNRFSLNLRSVTAADTAVYWCARSASIRGWFDPWGQGTLVTVSS

SEQ ID NO: 6 - protein, 104 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLP

SEQ ID NO: 7 - protein, 112 aa, Homo sapiens
VIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVP

SEQ ID NO: 8 - protein, 106 aa, Homo sapiens
DQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPK

SEQ ID NO: 9 - protein, 131 aa, Homo sapiens
GVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 10 - DNA, 337 bp, Homo sapiens
gacatccagatgacccagtctccatcctccttgtctgcgtctgtaggagacagagtcaccatcacttgccgggcaagtcagagtattagtagttatctaaattggtatcagcagaaaccagggaaagcccctaaggtcctgatctatgctgcatccactttgcgaagtggggtcccgtcaaggttcagtggcagtggatctgggacagatttcactctcaccgtcagcagtctgcaacctgaagattttgcaacttactactgtcaacagagttaccataccccgtggacgttcggtcaggggaccaaggtggaaatcaaacgaactgtggctgcac

SEQ ID NO: 11 - DNA, 369 bp, Homo sapiens
caggtgcagctgcaggagtcgggtccaggactgctgaagccttcggagaccctgtccctcacatgcactgtctctggtggctccgtcagtctttactactggagctggatccggcagtccccagggaaggaaccggagtggattggatatatctattccagtggaagcaccgattacaacccttccctcaggagtcgagtcaccatatcactggacacgtcaaacaaccggttttccctaaacctgaggtctgtgaccgccgcagatacagcggtctattggtgtgcgagaagtgcgtcaattaggggctggttcgacccctggggccagggaaccctggtcaccgtctcctcagggagtgcatccgcc

SEQ ID NO: 12 - DNA, 20 bp, Artificial Sequence
  Synthetically generated oligonucleotide
tccatgacgttcctgacgtt

SEQ ID NO: 13 - protein, 9 aa, Artificial Sequence
  Synthetically generated peptide
TYVPANASL

SEQ ID NO: 14 - DNA, 26 bp, Artificial Sequence
  Primer
ctacttcaagtaccacaatatgcgac

SEQ ID NO: 15 - DNA, 26 bp, Artificial Sequence
  Primer
cgttggctcctcttcttgcgaaactc

SEQ ID NO: 16 - DNA, 26 bp, Artificial Sequence
  Primer
cgtctggacttggctggccgggacct

SEQ ID NO: 17 - DNA, 26 bp, Artificial Sequence
  Primer
agtggccatctcctgctcgaagtcta

(19) United States
(12) Patent Application Publication
(10) Pub. No.: US 2010/0297146 A1
(43) Pub. Date: Nov. 25, 2010

(54) IMMUNE SYSTEM PROGRAMMING THROUGH B7-DC

(75) Inventor: Larry R. Pease, Rochester, MN (US)

Correspondence Address: FISH & RICHARDSON P.C., PO BOX 1022, MINNEAPOLIS, MN, 55440-1022, US

(73) Assignee: Mayo Foundation for Medical Education and Research a corporation

(21) Appl. No.: 12/444,820

(22) PCT Filed: Oct. 19, 2007
(86) PCT No.: PCT/US2007/081975

§ 371 Date: Jun. 30, 2010

Related U.S. Application Data
(60) Provisional application No. 60/862,306, filed on Oct. 20, 2006.

(51) Int. Cl. A61K 39/395; C12N 5/0784; A61P 37/02

(52) U.S. Cl. 424/172.1; 435/325

(57) ABSTRACT

Materials and methods related to modulating immune responses (e.g., altering the polarity of immune responses) are provided.

### CROSS-REFERENCE TO RELATED APPLICATIONS

This application claims benefit of U.S. Provisional Application Ser. No. 60/862,306, filed on Oct. 20, 2006.

### TECHNICAL FIELD

This document relates to materials and methods for altering the polarity of T-cell responses.

### BACKGROUND

The human IgM antibody B7-DC XAb (also referred to as B7-DC XAb, rHIgM12, or Lym12) can be used to switch the polarity (e.g., from a Th2 response to a Th1 response) of immune responses in vivo (Radhakrishnan et al. (2004) J. Immunol. 173:1360-1365; Radhakrishnan et al. (2005) J. Allergy Clin. Immunol. 116:668-674; and Van Keulen et al. (2005) Clin. Exp. Immunol. 143:314-321) or in vitro. To alter an immune response, antigen presenting cells can be pulsed with antigen at the time of B7-DC XAb treatment. Antigen can be pulsed in vivo or in vitro. However, there are some circumstances where the antigen in question may not be known or not available, and still other cases where administration of antigen or antigen-pulsed cells could be dangerous to the individual being treated (a patient with peanut allergy, for example).

### SUMMARY

The invention provides methods for modulating the polarity of immune responses without administering the antigen in question. Interactions between B7-DC XAb-activated dendritic cells (DC) and T-cells usually occur via MHC-mediated antigen recognition by T-cell receptors. The methods provided herein can include substituting this interaction with a surrogate communication bridge comprised of B7-DC XAb-activated DC coated via their Fc receptors with antibodies specific for components of the T-cell receptor complex.

In one aspect, this document features a method for modulating a state of immune responsiveness in a mammal. The method can include administering to the mammal an activated dendritic cell activated contacted by (a) a B7-DC cross-linking molecule, and (b) an antibody directed to a component of the T-cell receptor complex. The B7-DC cross-linking molecule can be an IgM antibody (e.g., an IgM antibody that recognizes a B7-DC epitope comprising a glycosylation site). The B7-DC crosslinking molecule can include an amino acid sequence that is between 80.0% and 99.9% identical (e.g., at least 95.0% identical, 99.1% identical, or 99.2% identical) to the amino acid sequence set forth in SEQ ID NO:3 or SEQ ID NO:5. The B7-DC crosslinking molecule can include an amino acid sequence that is between 80.0% and 99.9% identical to the amino acid sequence set forth in SEQ ID NO:3 or SEQ ID NO:5, and can further include an amino acid sequence that is at least 80.0% identical to the amino acid sequence set forth in SEQ ID NO:4, SEQ ID NO:6, SEQ ID NO:7, SEQ ID NO:8, or SEQ ID NO:9. The B7-DC cross-linking molecule can be B7-DC XAb. The antibody directed to a component of the T-cell receptor complex can be anti-CD3 antibody. The antibody directed to the T-cell receptor complex can contact an Fc receptor of the dendritic cell. The administering can be intravenous.

In another aspect, this document features a method for inhibiting tumor growth in a mammal having or at risk for having a tumor. The method can include administering to the mammal an activated dendritic cell contacted by (a) a B7-DC cross-linking molecule and (b) an antibody directed to a component of the T-cell receptor complex. The B7-DC cross-linking molecule can be an IgM antibody (e.g., an IgM antibody that recognizes a B7-DC epitope comprising a glycosylation site). The B7-DC crosslinking molecule can include an amino acid sequence that is between 80.0% and 99.9% identical (e.g., at least 95.0% identical, 99.1% identical, or 99.2% identical) to the amino acid sequence set forth in SEQ ID NO:3 or SEQ ID NO:5. The B7-DC crosslinking molecule can include an amino acid sequence that is between 80.0% and 99.9% identical to the amino acid sequence set forth in SEQ ID NO:3 or SEQ ID NO:5, and can further include an amino acid sequence that is at least 80.0% identical to the amino acid sequence set forth in SEQ ID NO:4, SEQ ID NO:6, SEQ ID NO:7, SEQ ID NO:8, or SEQ ID NO:9. The B7-DC cross-linking molecule can be B7-DC XAb. The antibody directed to a component of the T-cell receptor complex can be anti-CD3 antibody. The antibody directed to the T-cell receptor complex can contact an Fc receptor of the dendritic cell. The administering can be intravenous.

In another aspect, this document features a composition containing B7-DC XAb-activated dendritic cells contacted by an antibody directed to a component of the T-cell receptor complex. The B7-DC cross-linking molecule can be an IgM antibody (e.g., an IgM antibody that recognizes a B7-DC epitope comprising a glycosylation site). The B7-DC cross-linking molecule can be B7-DC XAb. The antibody directed to a component of the T-cell receptor complex is an anti-CD3 antibody. The antibody directed to the T-cell receptor complex can contact an Fc receptor of the dendritic cell.

In still another aspect, this document features a method for modulating regulatory T cell (Treg) function, comprising contacting a Treg with a B7-DC cross-linking molecule. The method can further include contacting the Treg with an antigen. The B7-DC cross-linking molecule can be an IgM antibody (e.g., an IgM antibody that recognizes a B7-DC epitope comprising a glycosylation site). The B7-DC crosslinking molecule can include an amino acid sequence that is between 80.0% and 99.9% identical (e.g., at least 95.0% identical, 99.1% identical, or 99.2% identical) to the amino acid sequence set forth in SEQ ID NO:3 or SEQ ID NO:5. The B7-DC crosslinking molecule can include an amino acid sequence that is between 80.0% and 99.9% identical to the amino acid sequence set forth in SEQ ID NO:3 or SEQ ID NO:5, and can further include an amino acid sequence that is at least 80.0% identical to the amino acid sequence set forth in SEQ ID NO:4, SEQ ID NO:6, SEQ ID NO:7, SEQ ID NO:8, or SEQ ID NO:9. The B7-DC cross-linking molecule can be B7-DC XAb. The antibody directed to a component of the T-cell receptor complex can be anti-CD3 antibody. The antibody directed to the T-cell receptor complex can contact an Fc receptor of the dendritic cell.

This document also features a method for modulating Treg function in a mammal, the method comprising administering to the mammal an activated dendritic cell contacted by (a) a B7-DC cross-linking molecule, and (b) an antibody directed to a component of the T-cell receptor complex. The method can further include contacting the Treg with an antigen. The B7-DC cross-linking molecule can be an IgM antibody (e.g., an IgM antibody that recognizes a B7-DC epitope comprising a glycosylation site). The B7-DC crosslinking molecule can include an amino acid sequence that is between 80.0% and 99.9% identical (e.g., at least 95.0% identical, 99.1% identical, or 99.2% identical) to the amino acid sequence set forth in SEQ ID NO:3 or SEQ ID NO:5. The B7-DC crosslinking molecule can include an amino acid sequence that is between 80.0% and 99.9% identical to the amino acid sequence set forth in SEQ ID NO:3 or SEQ ID NO:5, and can further include an amino acid sequence that is at least 80.0% identical to the amino acid sequence set forth in SEQ ID NO:4, SEQ ID NO:6, SEQ ID NO:7, SEQ ID NO:8, or SEQ ID NO:9. The B7-DC cross-linking molecule can be B7-DC XAb. The antibody directed to a component of the T-cell receptor complex can be anti-CD3 antibody. The antibody directed to the T-cell receptor complex can contact an Fc receptor of the dendritic cell. The administering can be intravenous.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a map of an expression vector that can be used to produce antibodies.

FIG. 2 is a schematic of an experimental protocol for altering and assessing the polarity of immune responses in spleen cells from OVA-immunized mice.

FIGS. 3A and 3B are graphs plotting cytokine levels in culture supernatants of spleen cells from mice immunized with OVA. Spleen cells were harvested and incubated for 48 hours with the B7-DC XAb IgM antibody or the s39 isotype control IgM antibody, as indicated, and culture supernatants were assayed for levels of IFNγ (FIG. 3A) and IL-4 (FIG. 3B).

FIG. 4 is a schematic of an experimental protocol for altering and assessing the polarity of immune responses in spleen cells from OVA-immunized mice.

FIGS. 5A and 5B are graphs plotting Th2 recall responses in spleen cells supplemented with exogenous DC. FIG. 5A shows IL-4 levels in culture media from spleen cells treated directly with control or B7-DC XAb IgM antibodies as depicted in FIG. 1. FIG. 5B shows IL-4 levels in culture media of spleen cells incubated with DC activated with s39 control or B7-DC XAb, as indicated, with or without the recall OVA antigen (Ag), as indicated, and with or without coating with CD3 antibody, as indicated.

FIGS. 6A and 6B are graphs plotting Th1 recall responses in spleen cells supplemented with exogenous DC. FIG. 6A shows IFNγ levels in culture media from spleen cells treated directly with control or B7-DC XAb IgM antibodies as depicted in FIG. 1. FIG. 6B shows IFNγ levels in culture media of spleen cells incubated with DC activated with s39 control or B7-DC XAb, as indicated, with or without the recall OVA antigen (Ag), as indicated, and with or without coating with CD3 antibody, as indicated.

FIGS. 7A and 7B are graphs plotting IL-4 (FIG. 7A) and IFNγ (FIG. 7B) levels in culture supernatants from primed spleen cells incubated with CD3-coated DC that had been activated by s39 or B7-DC XAb. In addition, OVA antigen was added to the cultures at day 0, day 1, or day 2 after addition of DC, or not at all, as indicated.

FIG. 8 is a graph plotting survival of mice that were injected subcutaneously with B16 melanoma cells, and treated systemically with s39 control IgM (filled squares), B7-DC Xab (filled triangles), or CD3 antibody-coated DC that had been activated with s39 (filled circles) or B7-DC XAb (open squares).

FIGS. 9A and 9B are graphs plotting IL-4 levels (FIG. 9A) and IFN-γ levels (FIG. 9B) in spleen cells stimulated with OVA in the presence of control IgM Ab, B7-DC XAb, CpG, pI:C, or anti-CD40. Other controls shown are supernatants from naive splenocytes stimulated with OVA alone, cytokine secretion in absence of Ag, and stimulation of spleen cells with Ag plus an IgG Ab (clone TY-25) that binds to B7-DC. All data are based on supernatant samples from triplicate cultures.

FIG. 10A is a schematic of an experimental protocol for restimulating spleen cells polarized toward a Th2 phenotype in vitro with DC treated with control IgM Ab or B7-DC XAb. FIGS. 10B and 10C are graphs plotting levels of IL-4 (FIG. 10B) and IFN-γ (FIG. 10C) in restimulated spleen cells. FIGS. 10D and 10E are graphs similar to those shown in FIGS. 10B and 10C, except that the presensitized splenocytes were incubated with DC treated with various agonists, including TLR ligands or anti-CD40, as indicated. FIGS. 10F and 10G are graphs plotting IL4 and IFN-γ levels, respectively, from spleen cells stimulated with DC that were pretreated with 20 μg/ml (filled bars), 10 μg/ml (open bars), or 5 μg/ml (shaded bars) TLR9 agonist CpG or with the TLR7/8 agonist Gardiquimod.

FIG. 11 is a series of graphs showing that B7-DC XAb induces Th1 cells, whereas TLR ligation yields Th0 cells in splenic recall responses. Th2 primed splenocytes were restimulated with Ag-pulsed DC that were treated with control IgM Ab, B7-DC XAb, anti-CD40, pI:C, CpG, LPS, or Gardiquimod, as indicated. The spleen cells were permeabilized and stained for CD4, IL-4, and IFN-γ. Intracellular cytokine levels were assessed on CD4+ cells by flow cytometry.

FIG. 12 is a series of graphs showing phenotype reversal of purified Th2-polarized T cells by enriched DC populations. CD11c⁻, CD11b⁺, Gr-1^{int}, and CD3⁻ cells were isolated from GM-CSF, IL-4-supplemented bone marrow cultures treated with control IgM Ab (FIG. 12A) or B7-DC XAb (not shown) plus Ag. CD11c⁻ cells in the enriched fraction were CD11b+CD3⁻. Highly enriched splenic T cells isolated from OVA/alum immunized mice were >98% CD3⁺ (FIG. 12B). Cytokine levels were assessed in culture supernatants following co-culture of the enriched DC and T cell populations (FIGS. 12C and 12D). Enriched DC from cultures pulsed with sperm whale myoglobulin treated with control IgM Ab or B7-DC XAb were coincubated with a CD4⁺ sperm whale myoglobulin-specific cloned T cell line (FIGS. 12E and 12F).

FIG. 13 is a series of graphs showing that B7-DC XAb-mediated phenotype reversal is restricted by appropriate MHC molecules and is Ag specific. FIGS. 13A and 13B are graphs plotting levels of IL-4 and IFN-γ, respectively, in spleen cells from Th2-polarized mice that were restimulated in vitro with Ag-pulsed, control IgM Ab-treated syngenic DC. FIG. 13C is a graph depicting the proliferative response of Th2-polarized T cells to allogenic DC in a MLR assessed by the incorporation of [³H]thymidine. Open symbols represent the response of splenocytes to syngenic DC, while filled symbols represent the response of splenocytes to allogenic DC. FIGS. 13D and 13E are graphs depicting the inability of Ag pulsed, control IgM, or B7-DC XAb-treated DC to modulate the polarity of cytokine production of Th2-polarized, OVA-specific T cells. WT, wild type. FIGS. 13F to 13I are graphs plotting levels of IL-4 and IFN-γ, as indicated, in cultures presensitized to OVA or to BSA, as indicated. Splenocytes were restimulated with the appropriate Ag along with OVA-pulsed DC treated with isotype control IgM Ab (open circles) or B7-DC XAb (filled circles) or with BSA pulsed DC treated with isotype control IgM Ab (open squares) or B7-DC XAb (filled squares).

FIG. 14 is a series of graphs indicating the importance of SLAM and ICAM-1 for B7-DC XAb-induced phenotype reversal. Wild-type Th2-polarized spleen cells were stimulated with Ag-pulsed DC that were wild type (circles) or SLAM deficient (squares) (FIGS. 14A and 14B) or wild type (circles) or ICAM-1 deficient (squares) (FIGS. 14C and 14D). Culture supernatants were assayed for IL-4 (FIGS. 14A and 14C) or IFN-γ (FIGS. 14B and 14D). Open symbols represent DC treated with 10 μg/ml isotype control Ab, while closed symbols represent DC treated with 10 μg/ml B7-DC XAb. FIGS. 14E and 14F are similar to 14C and 14D, except that wild-type DC or DC deficient for B7.1 and B7.2 were tested for the ability to modulate recall response polarity of the presensitized spleen cell cultures.

FIGS. 15A to 15D are graphs depicting IL-4 and IFN-γ production by Th2-polarized spleen cells that were stimulated with wild-type DC derived from cytokine-deficient KO mice. The DC were pulsed with Ag and treated with either control IgM Ab or B7-DC XAb before addition to the spleen cell cultures. FIGS. 15A and 15B are similar to 15C and 15D, except that IL-12-deficient DC (15C and 15D) were used to stimulate the Th2 spleen cells instead of IFN-y^{-/-} DC (15A and 15B). FIGS. 15E and 15F are similar to 15G and 15H, except that STAT4- and STAT6-deficient DC (15E and 15F) were used to stimulate Th2 spleen cells instead of IFN-γ receptor DC (15G and 15H). FIGS. 15G and 15H depict IL-4 and IFN-γ responses from wild-type or IFN-γ receptor-deficient (IFN-γR^{-/-}) Th2-polarized splenocytes that were stimulated with Ag-pulsed wild-type DC treated with control IgM Ab or B7-DC XAb.

FIG. 16 depicts immunization strategies as used in the Treg experiments described herein. In the strategy depicted in FIG. 16A, Balb/c mice that are transgenic for ratHer2neu were subjected to Treg cell depletion by injecting anti-CD25 antibody twice prior to first injection of CpG. Control peptide or immunogenic peptide were immunized on 3 day of CpG injection. Mice were challenged with TUBO tumor and were later used to monitor the T cell response. The strategy depicted in FIG. 16B is similar to protocol A, except that no Treg depleting antibody was administered. Mice received three injections of B7-DC XAb 1 day before, the day of, and 1 day later after immunization with peptide.

FIG. 17 is a pair of graphs plotting reversal of T cell tolerance achieved by Treg depletion or by treatment with B7-DC XAb. Mice expressing rat Her2neu as self antigen were immunized with a known immunogenic peptide (p66) from Her2neu or control peptide from Plasmodium yoelii along with CpG treatment or concomitant depletion of Treg depletion (FIG. 17A) or in combination with B7-DC XAb treatment (FIG. 17B). Following challenge with TUBO tumor, CD8 T cell response was monitored by their ability to make IFNγ measured by ELISPOT when stimulated with P815 cells pulsed with p66 peptide (open bars), parental tumor, TUBO, (filled bars), a mouse breast cancer cell line transfected with rat Her2neu, A2L2, (hatched bars) or mock transfected cells, 66.3neo (dotted bars). All measurements were done in triplicate.

FIG. 18A is a graph plotting FoxP3 message levels in non-Tregs and Tregs isolated from DO11.10 TCR transgenic T cells and stimulated with antigen-pulsed control or B7-DC XAb treated dendritic cells. All groups of cells were analyzed for FoxP3 message by RT-PCR. FIG. 18B is a series of graphs depicting the intracellular profile of FoxP3 on CD4 positive cells on non Tregs (top) and Tregs (middle) that were stimulated as described above. The bottom pair of FIG. 18B shows the status of FoxP3 on Tregs that were not in contact with antigen-pulsed antibody treated dendritic cells.

FIG. 19 is a series of graphs depicting the lack of FoxP3 down regulation by B7-DC XAb in the absence of antigen. Tregs from D011.10 mice were stimulated with control antibody or B7-DC XAb dendritic cells and not pulsed with ovalbumin. Graphs on the left represent the control antibody treated group, and those on the right represent B7-DC XAb treated group.

FIG. 20 is a series of graphs depicting the kinetics of FoxP3 downregulation mediated by B7-DC XAb. Purified Tregs from D011.10 mice were stimulated with dendritic cells pulsed with antigen and treated with control antibody or B7-DC XAb and were monitored for FoxP3 levels. The graphs in FIG. 20A indicate FoxP3 expression in clonotypic antibody gated (bottom left), CD4 positive cells (top group), while comparison of FoxP3 levels between control antibody (bottom group, filled histogram) and B7-DC XAb treated DC (bottom group, open histogram) is shown as histograms. FIGS. 20B and 20C are similar to FIG. 20A except that the time points are 36 hours and 48 hours, respectively.

FIG. 21 is a pair of graphs indicating that TLR9 ligation on dendritic cells does not lead to down regulation of FoxP3 in Tregs. DO11.10 Tregs were isolated and stimulated as in FIG. 20, except CpG was used to stimulated dendritic cells. Control antibody treatment was used as negative control. CD4 positive cells were stained for FoxP3 after 48 hours.

FIG. 22 is a series of graphs showing that B7-DC XAb induces antigen specific down regulation of FoxP3. FIG. 22A shows a FoxP3 expression profile using Tregs isolated from DO11.10 mice were mixed with Tregs isolated from Balb/C mice. Dendritic cells from Balb/C mice that were ovalbumin pulsed and control antibody or B7-DC XAb treated was used as stimulators. Expression of FoxP3 was monitored by flow cytometry. Cells were gated through clonotypic antibody (KJ1-26) positive cells (D011.10 Tregs) (top group) or negative cells (Balb/C Tregs) (bottom group). FIG. 22B shows a FoxP3 expression profile on CD4 positive cells from the spleens of mice injected with D011.10 Tregs along with ovalbumin and control antibody or B7-DC XAb after 48 hours. The top group represents cells that are KJ1-26 positive and bottom group represents cells that are KJ1-26 negative.

FIG. 23 is a series of graphs indicating that dendritic cells treated with B7-DC XAb convert Tregs to effector cells. DO11.10 non Tregs and Tregs were stimulated with antigen-pulsed control or B7-DC XAb treated dendritic cells, and cell proliferation in response to antigen was measured by the amount of [³H] incorporation. In the left panel of FIG. 23A, open symbols indicate the proliferative Treg response to control antibody treated dendritic cells, while filled symbols indicate the response of non-Tregs stimulated with B7-DC XAb activated DC. To study the ability of B7-DC XAb treated DC to modulate Treg function, a mixture of Tregs and non Tregs was stimulated with ovalbumin pulsed control antibody treated DC, (FIG. 23A, right panel, open symbols) or with ovalbumin pulsed B7-DC XAb treated DC, (right panel, filled symbols). Cytokine profiles of CD4 positive Tregs were verified after stimulation of the indicated cell population as in FIG. 18. Cells were permeabilized and stained for IFNγ (FIG. 23B, top left pair), IL-17 (top right pair), TNFα (bottom left pair), and IL-10 (bottom right pair). FIG. 23C is a graph showing the level of TFGβ1 secretion of the indicated cell types, as determined by ELISA.

FIG. 24 is a series of graphs showing that FoxP3 down regulation occurs independent of cell division. Tregs were labeled with CFSE and stimulated as in FIG. 18. Surface staining for CD4 was followed by intracellular staining for FoxP3. FoxP3 and CFSE profiles of the indicated groups are depicted in the top panels, while the bottom panels show histogram profiles comparing control antibody stimulated Tregs (filled histograms) versus B7-DC XAb stimulated Tregs (open histograms) for the indicated markers.

FIG. 25 is a series of graphs indicating that IL-6 is crucial for B7-DC XAb induced down regulation of FoxP3. The graphs in FIG. 25A plot FoxP3 levels on CD4 positive OT-II cells stimulated with dendritic cells from wild type mice or IL-6^{-/-} mice that were pulsed with ovalbumin and treated with control antibody or B7-DC XAb. The graphs in FIG. 25B are similar to those in FIG. 25A, except that CFSE labeled OT-II Tregs were adoptively transferred into wild type or IL-6^{-/-} mice along with ovalbumin and control antibody or B7-DC XAb.

FIG. 26 is a series of graphs and pictures indicating that induction of autoimmune diabetes by B7-DC XAb is dependent on IL-6. RIP-Ova mice were injected with ovalbumin pulsed control antibody or B7-DC XAb treated wild type or IL-6^{-/-} dendritic cells. The graphs in FIG. 26A plot blood glucose levels in the treated mice. When the sugar levels reached 500 mg/dl or more, mice from all groups were sacrificed, and the pancreas were isolated and fixed in formalin. Tissues were processed and analyzed for inflammation (FIG. 26B, top row), T cell infiltration (middle row), and the presence of β-cells as verified by staining for insulin (bottom row).

FIG. 27 is a pair of graphs indicating that IL-6 is dispensable for B7-DC XAb induced generation of anti tumor CTL. Wild type mice or IL-6^{-/-} mice were injected with B16 tumor cells and treated with control antibody or B7-DC XAb. On day 7, draining lymph node cells were used as effectors to lyse B16 tumor cell targets (left panel). EL4 tumor cell target were used as negative control (right panel). Filled circles and filled squares represent effectors derived from B7-DC XAb treated wild type or IL-6^{-/-} mice, respectively. Open squares and open circles represent effectors derived from control antibody treated wild type or IL-6^{-/-} mice, respectively.

### B7-DC XAb SEQUENCES

SEQ ID NO:1—amino terminal sequence of the B7-DC XAb heavy chain

Val-Gln-Leu-Gln-Glu-Ser-Gly-Pro-Gly-Leu-Leu-Lys-
Pro-Ser-Glu-Thr-Leu-Arg/Ser-Leu-Thr-Asn

SEQ ID NO:2—amino terminal sequence of the B7-DC XAb light chain

Asp-Ile-Gln-Met-Thr-Gln-Ser-Pro-Ser-Ser-Leu-Ser-
Ala-Ser-Val-Gly-Asp-Arg-Val

SEQ ID NO:3—variable (Vk) domain of the B7-DC XAb light chain

DIQMTQSPSSLSASVGDRVTITCRASQSISSYLNWYQQKPGKAPKVLIYA
ASLRSGVPSRFSGSGSGTDFTLTVSSLQPEDFATYYCQQSYHTPWTFGQG
TKVEIK

SEQ ID NO:4—constant (Ck) domain of the B7-DC XAb light chain

RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

SEQ ID NO:5—variable (Vh) domain of the B7-DC XAb heavy chain

QVQLQESGPGLLKPSETLSLTCTVSGGSVSLYYWSWIRQSPGKEPEWIGY
IYSSGSTDYNPSLRSRVTISLDTSNNRFSLNLRSVTAADTAVYWCARSAS
IRGWFDPWGQGTLVTVSS

SEQ ID NO:6—constant (CH1) domain of the B7-DC XAb heavy chain

GSASAPTLFPLVSCENSPSDTSSVAVGCLAQDFLPDSITFSWKYKNNSDI
SSTRGFPSVLRGGKYAATSQVLLPSKDVMQGTDEHVVCKVQHPNGNKEKN
VPLP

SEQ ID NO:7—constant (CH2) domain of the B7-DC XAb heavy chain

VIAELPPKVSVFVPPRDGFFGNPRKSKLICQATGFSPRQIQVSWLREGKQ
VGSGVTTDQVQAEAKESGPTTYKVTSTLTIKESDWLSQSMFTCRVDHRGL
TFQQNASSMCVP

SEQ ID NO:8—constant (CH3) domain of the B7-DC XAb heavy chain

DQDTAIRVFAIPPSFASIFLTKSTKLTCLVTDLTTYDSVTISWTRQNGEA
VKTHTNISESHPNATFSAVGEASICEDDWNSGERFTCTVTHTDLPSPLKQ
TISRPK

SEQ ID NO:9—constant (CH4) domain of the B7-DC XAb heavy chain

GVALHRPDVYLLPPAREQLNLRESATITCLVTGFSPADVFVQWMQRGQPL
SPEKYVTSAPMPEPQAPGRYFAHSILTVSEEEWNTGETYTCVVAHEALPN
RVTERTVDKSTGKPTLYNVSLVMSDTAGTCY

SEQ ID NO:10—DNA sequence encoding the Vk domains of B7-DC XAb

GACATCCAGATGACCCAGTCTCCATCCTCCTTGTCTGCGTCTGTAGGAGA
CAGAGTCACCATCACTTGCCGGGCAAGTCAGAGTATTAGTAGTTATCTAA
ATTGGTATCAGCAGAAACCAGGGAAAGCCCCTAAGGTCCTGATCTATGCT
GCATCCACTTTGCGAAGTGGGGTCCCGTCAAGGTTCAGTGGCAGTGGATC
TGGGACAGATTTCACTCTCACCGTCAGCAGTCTGCAACCTGAAGATTTTG
CAACTTACTACTGTCAACAGAGTTACCATACCCCGTGGACGTTCGGTCAG
GGGACCAAGGTGGAAATCAAACGAACTGTGGCTGCAC

SEQ ID NO:11—DNA sequence encoding the Vh domains of B7-DC XAb

CAGGTGCAGCTGCAGGAGTCGGGTCCAGGACTGCTGAAGCCTTCGGAGAC
CCTGTCCCTCACATGCACTGTCTCTGGTGGCTCCGTCAGTCTTTACTACT
GGAGCTGGATCCGGCAGTCCCCAGGGAAGGAACCGGAGTGGATTGGATAT
ATCTATTCCAGTGGAAGCACCGATTACAACCCTTCCCTCAGGAGTCGAGT
CACCATATCACTGGACACGTCAAACAACCGGTTTTCCCTAAACCTGAGGT
CTGTGACCGCCGCAGATACAGCGGTCTATTGGTGTGCGAGAAGTGCGTCA
ATTAGGGGCTGGTTCGACCCCTGGGGCCAGGGAACCCTGGTCACCGTCTC
CTCAGGGAGTGCATCCGCC

### DETAILED DESCRIPTION

### 1. B7-DC Cross-Linking Molecules

This document provides molecules that bind specifically to B7-DC polypeptides. Such molecules can bind simultaneously to a plurality of B7-DC polypeptides (i.e., one such molecule can bind to more than one B7-DC polypeptide at the same time). Molecules provided herein thus can effectively cross-link a plurality of B7-DC polypeptides. Methods for evaluating the binding of a molecule to B7-DC are disclosed in, for example, U.S. Patent Publication Nos. 2004/0014207, 2006/0002928, and 2006/0099203, which are incorporated herein by reference in their entirety.

The molecules provided herein typically are polypeptides, and antibodies can be particularly useful (see below), but other multivalent molecules that can bind and cross-link B7-DC on the surface of cells also can function in this capacity. Examples of such molecules include, without limitation, multivalent RNA or DNA aptamers. Aptamers typically are single-stranded DNA and RNA molecules that, like antibodies, can bind target molecules with affinity and specificity. Although nucleic acids are commonly thought of as linear molecules, they actually can take on complex, sequence-dependent, three-dimensional shapes. When the resulting shapes interact with a target protein, the result can be a tightly bound complex analogous to an antibody-antigen interaction. Aptamers can be modified to resist nuclease digestion, for example, or to enhance their therapeutic usefulness (e.g., to remain in the bloodstream longer, or to be stable in serum).

Molecules provided herein can bind specifically to cells through B7-DC polypeptides that are present on the cell surface. As used herein, “binds specifically” means that a molecule binds preferentially to a particular target molecule, and does not display significant binding to other relevant molecules (e.g., substantially less, or no, detectable binding to other molecules on, for example, immune system cells). For example, an antibody that binds specifically to a particular antigen binds preferentially to that antigen, and does not display significant binding to other relevant antigens. In an analogous manner, a molecule that “binds specifically to B7-DC” binds preferentially to B7-DC and does not display significant binding to other cell surface polypeptides of the human immune system. As disclosed herein, a polypeptide is an amino acid chain, regardless of post-translational modification. Thus, a molecule that binds specifically to a B7-DC polypeptide can “recognize” the B7-DC amino acid sequence or a portion thereof, a post-translational modification of B7-DC (e.g., one or more glycosylated or phosphorylated positions within the B7-DC amino acid sequence), or a combination thereof Molecules (e.g., antibodies or polypeptides) can be tested for recognition of B7-DC using standard immunoassay methods, including FACS, enzyme-linked immunosorbent assay (ELISA), and radioimmuno assay (RIA). See, e.g., Short Protocols in Molecular Biology, eds. Ausubel et al., Green Publishing Associates and John Wiley & Sons (1992).

B7-DC is a cell surface polypeptide that can be found on, for example, DC, activated macrophages, endothelial cells, and some tumor cells (e.g., glioma tumor cells). Molecules provided herein can bind to B7-DC on the surface of DC in a mammal (e.g., a human) and potentiate an immune response. As used herein, the term “potentiate an immune response” encompasses enhancement of DC function and increased activation of naïve T-cells. Enhanced DC function includes components such as prolonged longevity of DC, which can be detected based on increased expression of NF-κB and increased translocation of NF-κB to the nucleus. Other components of enhanced DC function include an increased ability of DC to activate naïve T-cells, increased localization of DC to the lymph nodes, increased phosphorylation of AKT (also known as protein kinase B) within DC, and increased secretion of cytokines such as interleukin-6 (IL-6), IL-12, RANTES, and tumor necrosis factor-alpha (TNF-α) by DC. Molecules provided herein also can enhance the metabolism of DC upon the withdrawal of cytokines from DC in culture. The molecules described herein can be administered to a mammal (e.g., a human) in order to enhance DC function and potentiate an immune response that can include any or all of the above-listed components. Such molecules also can be used to contact and activate DC in vitro.

Potentiation of an immune response can be measured by assessing any of the components listed above. Secretion of cytokines such as IL-12 can be measured, for example, by an enzyme linked immunosorbent (ELISA) assay as described in the Examples (below). Activation of naïve T-cells can be assayed by, for example, measuring the incorporation of ³H-thymidine into newly synthesized DNA in proliferating cells, measuring induction of cytolytic T-cell activity, or by detecting T-cell activation markers such as CD44 and/or CD69. Expression or translocation of NF-κB can be measured by, for example, cell staining with an antibody against NF-κB. Increased phosphorylation of AKT can be assessed by, for example, western blotting with an antibody against phosphorylated AKT. Antibodies against NF-κB and phosphorylated AKT are available from, for example, Cell Signaling Technologies, Inc. (Beverly, Mass.). Methods for measuring the other components encompassed by enhanced DC function and immunopotentiation also are described herein.

The molecules provided herein typically are purified. The term “purified” as used herein refers to a molecule that has been separated or isolated from other cellular components by which it is naturally accompanied (e.g., other cellular proteins, polynucleotides, or cellular components), or separated from most or all other components present in a reaction mixture when the molecule is synthesized in vitro. “Purified” as used herein also encompasses molecules that are partially purified, so that at least some of the components by which the molecule is accompanied are removed. Typically, a molecule is considered “purified” when it is at least 50% (e.g., 55%, 60%, 70%, 80%, 90%, 95%, or 99%), by dry weight, free from the proteins and other organic molecules or components with which it naturally associates or with which it is accompanied in a synthesis reaction.

Aptamers can be initially selected for specific binding activities from a starting pool of nucleic acids using, for example, methods known in the art. Variants can be obtained during subsequent rounds of amplification. An aptamer can be considered purified when it is at least 50% free from the other nucleic acids in the pool from which it is isolated.

### 2. Polypeptides and Antibodies

As used herein, a polypeptide is an amino acid chain, regardless of length or post-translational modification (e.g., phosphorylation or glycosylation). The polypeptides provided herein can bind specifically to B7-DC, and upon administration to a mammal (e.g., a human), can enhance DC function and potentiate an immune response. Polypeptides provided herein also can enhance DC function when incubated in vitro with DC.

The polypeptides featured herein can contain an amino acid sequence that is similar or identical to the amino sequence of B7-DC XAb. For example, a polypeptide can contain an amino acid sequence that is at least 80.0% identical (e.g., 80.0%, 85.0%, 90.0%, 95.0%, 95.5%, 96.0%, 96.5%, 97.0%, 97.5%, 98.0%, 98.5%, 99.0%, 99.1%, 99.2%, 99.3%, 99.4%, 99.5%, 99.6%, 99.7%, 99.8%, 99.9%, from 80.0% to 99.9%, from 95.0% to 99.9%, from 96.0% to 99.9%, from 97.0% to 99.9%, or from 98.0% to 99.9% identical) to the amino acid sequence set forth in SEQ ID NO:3 or SEQ ID NO:5. In some embodiments, a polypeptide can further contain an amino acid sequence that is at least 80.0% identical (e.g., 80.0%, 85.0%, 90.0%, 95.0%, 95.5%, 96.0%, 96.5%, 97.0%, 97.5%, 98.0%, 98.5%, 99.0%, 99.1%, 99.2%, 99.3%, 99.4%, 99.5%, 99.6%, 99.7%, 99.8%, 99.9%, from 80.0% to 99.9%, from 95.0% to 99.9%, from 96.0% to 99.9%, from 97.0% to 99.9%, or from 98.0% to 99.9% identical) to the amino acid sequence set forth in SEQ ID NO:4, SEQ ID NO:6, SEQ ID NO:7, SEQ ID NO:8, or SEQ ID NO:9. Percent sequence identity is calculated by determining the number of matched positions in aligned amino acid sequences, dividing the number of matched positions by the total number of aligned amino acids, and multiplying by 100. A matched position refers to a position in which identical amino acids occur at the same position in aligned amino acid sequences. Percent sequence identity also can be determined for any nucleic acid sequence.

To determine percent sequence identity, a target nucleic acid or amino acid sequence is compared to the identified nucleic acid or amino acid sequence using the BLAST 2 Sequences (Bl2seq) program from the stand-alone version of BLASTZ containing BLASTN version 2.0.14 and BLASTP version 2.0.14. This stand-alone version of BLASTZ can be obtained on the World Wide Web at fr.com/blast or at ncbi.nlm.nih.gov. Instructions explaining how to use the Bl2seq program can be found in the readme file accompanying BLASTZ.

Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: −i is set to a file containing the first nucleic acid sequence to be compared (e.g., C:\seq1.txt); −j is set to a file containing the second nucleic acid sequence to be compared (e.g., C:\seq2.txt); −p is set to blastn; −o is set to any desired file name (e.g., C:\output.txt); −q is set to −1; −r is set to 2; and all other options are left at their default setting. For example, the following command can be used to generate an output file containing a comparison between two sequences: C:\Bl2seq −i c:\seq1.txt −j c:\seq2.txt −p blastn −o c:\output.txt −q −1 −r 2. To compare two amino acid sequences, the options of Bl2seq are set as follows: −i is set to a file containing the first amino acid sequence to be compared (e.g., C:\seq1.txt); −j is set to a file containing the second amino acid sequence to be compared (e.g., C:\seq2.txt); −p is set to blastp; −o is set to any desired file name (e.g., C:\output.txt); and all other options are left at their default setting. For example, the following command can be used to generate an output file containing a comparison between two amino acid sequences: C:\Bl2seq −i c:\seq1.txt −j c:\seq2.txt −p blastp −o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence (e.g., SEQ ID NO:3), or by an articulated length (e.g., 100 consecutive nucleotides or amino acid residues from a sequence set forth in an identified sequence), followed by multiplying the resulting value by 100. For example, a nucleic acid sequence that has 98 matches when aligned with the sequence set forth in SEQ ID NO:3 is 92.5 percent identical to the sequence set forth in SEQ ID NO:3 (i.e., 98±106*100=92.5). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It is also noted that the length value will always be an integer.

The amino acid sequences of the polypeptides provided herein can have substitutions, deletions, or additions with respect to the amino acid sequences set forth in SEQ ID NOS:3 and 5. A polypeptide having an amino acid sequence that is modified (e.g., by substitution) with respect to SEQ ID NO:3 and/or SEQ ID NO:5 can have substantially the same or improved qualities as compared to a polypeptide containing the amino acid sequence identical to that set forth in SEQ ID NO:3 and SEQ ID NO:5. A substitution can be a conserved substitution. As used herein, a “conserved substitution” is a substitution of an amino acid with another amino acid having a similar side chain. A conserved substitution typically can be a substitution with an amino acid that makes the smallest change possible in the charge of the amino acid or size of the side chain of the amino acid (alternatively, in the size, charge or kind of chemical group within the side chain) such that the overall peptide essentially retains its spatial conformation but has altered biological activity. Examples of conserved changes include, without limitation, Asp to Glu, Asn or Gln; His to Lys, Arg or Phe; Asn to Gln, Asp or Glu, and Ser to Cys, Thr or Gly. Alanine is commonly used to substitute for other amino acids. The 20 essential amino acids can be grouped as follows: alanine, valine, leucine, isoleucine, proline, phenylalanine, tryptophan and methionine having nonpolar side chains; glycine, serine, threonine, cysteine, tyrosine, asparagine and glutamine having uncharged polar side chains; aspartate and glutamate having acidic side chains; and lysine, arginine, and histidine having basic side chains (see, e.g., Stryer, Biochemistry (2^{nd} edition) W. H. Freeman and Co. San Francisco (1981), pp. 14-15; and Lehninger, Biochemistry (2^{nd} edition, 1975), pp. 73-75). Conservative substitutions can include substitutions made within these groups.

PD-1 is a polypeptide that is a natural receptor for B7-DC. PD-1 can be immobilized on a solid substrate (e.g., a plastic dish or a glass microscope slide). Upon incubation with DC, immobilized PD-1 can cross-link a plurality of B7-DC polypeptides on the cell surface and enhance the function of the DC. Incubation of cultured DC with immobilized PD-1 can, for example, maintain the metabolic rate of the cells upon removal of cytokines from the culture medium, as compared to the metabolic rate of DC that are not incubated with PD-1.

Molecules provided herein can be antibodies that have specific binding activity for B7-DC. The terms “antibody” and “antibodies” encompass intact molecules as well as fragments thereof that are capable of binding to B7-DC. Antibodies can be polyclonal antibodies, monoclonal antibodies, humanized or chimeric antibodies, single chain Fv antibody fragments, Fab fragments, and F(ab)₂ fragments. Polyclonal antibodies are heterogeneous populations of antibody molecules that are specific for a particular antigen, while monoclonal antibodies are homogeneous populations of antibodies to a particular epitope contained within an antigen.

An antibody can be of any immunoglobulin (Ig) class, including IgM, IgA, IgD, IgE, and IgG, and any subclass thereof. Antibodies of the IgM class (e.g., B7-DC XAb) typically are pentavalent and are particularly useful because one antibody molecule can cross-link a plurality of B7-DC polypeptides. Immune complexes containing Ig molecules that are cross-linked (e.g., cross-linked IgG) and are thus multivalent also are capable of cross-linking a plurality of B7-DC molecules, and can be particularly useful.

As used herein, an “epitope” is a portion of an antigenic molecule to which an antibody binds. Antigens can present more than one epitope at the same time. For polypeptide antigens, an epitope typically is about four to six amino acids in length, and can include modified (e.g., phosphorylated or glycosylated) amino acids. Two different immunoglobulins can have the same epitope specificity if they bind to the same epitope or set of epitopes.

Polyclonal antibodies are contained in the sera of immunized animals. Monoclonal antibodies can be prepared using, for example, standard hybridoma technology. In particular, monoclonal antibodies can be obtained by any technique that provides for the production of antibody molecules by continuous cell lines in culture as described, for example, by Kohler et al. (1975) Nature 256:495-497, the human B-cell hybridoma technique of Kosbor et al. (1983) Immunology Today 4:72, and Cote et al. (1983) Proc. Natl. Acad. Sci. USA 80:2026-2030, and the EBV-hybridoma technique of Cole et al., Monoclonal Antibodies and Cancer Therapy, Alan R. Liss, Inc. pp. 77-96 (1983). A hybridoma producing monoclonal antibodies can be cultivated in vitro or in vivo.

Antibodies such as those provided herein also can be isolated from, for example, the serum of an individual. The B7-DC XAb antibody, for example, was isolated from human serum as described in Example 1 herein. Suitable methods for isolation include purification from mammalian serum using techniques that include, for example, chromatography.

Antibodies that bind to B7-DC also can be produced by, for example, immunizing host animals (e.g., rabbits, chickens, mice, guinea pigs, or rats) with B7-DC. A B7-DC polypeptide or a portion of a B7-DC polypeptide can be produced recombinantly, by chemical synthesis, or by purification of the native protein, and then used to immunize animals by injection of the polypeptide. Adjuvants can be used to increase the immunological response, depending on the host species. Suitable adjuvants include Freund's adjuvant (complete and incomplete), mineral gels such as aluminum hydroxide, surface active substances such as lysolecithin, pluronic polyols, polyanions, peptides, oil emulsions, keyhole limpet hemocyanin (KLH), and dinitrophenol. Standard techniques can be used to isolate antibodies generated in response to the B7-DC immunogen from the sera of the host animals. Such techniques are useful for generating antibodies that have similar characteristics to B7-DC XAb (e.g., similar epitope specificity and other functional similarities).

Antibodies such as B7-DC XAb also can be produced recombinantly. The amino acid sequence (e.g., the partial amino acid sequence) of an antibody provided herein can be determined by standard techniques, and a cDNA encoding the antibody or a portion of the antibody can be isolated from the serum of the subject (e.g., the human patient or the immunized host animal) from which the antibody was originally isolated. The cDNA can be cloned into an expression vector using standard techniques. The expression vector then can be transfected into an appropriate host cell (e.g., a Chinese hamster ovary cell, a COS cell, or a hybridoma cell), and the antibody can be expressed and purified.

Antibody fragments that have specific binding affinity for B7-DC and retain cross-linking function also can be generated by techniques such as those disclosed above. Such antibody fragments include, but are not limited to, F(ab′)₂ fragments that can be produced by pepsin digestion of an antibody molecule, and Fab fragments that can be generated by reducing the disulfide bridges of F(ab′)₂ fragments. Alternatively, Fab expression libraries can be constructed. See, for example, Huse et al. (1989) Science 246:1275-1281. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques, such as those disclosed in U.S. Pat. No. 4,946,778. Such fragments can be rendered multivalent by, for example, biotinylation and cross-linking, thus generating antibody fragments that can cross-link a plurality of B7-DC polypeptides.

### 3. Nucleic Acids, Vectors, and Host Cells

This document provides nucleic acids encoding molecules (e.g., polypeptides and antibodies such as those described herein) that bind specifically to B7-DC. As used herein, the term “nucleic acid” refers to both RNA and DNA, including cDNA, genomic DNA, and synthetic (e.g., chemically synthesized) DNA. A nucleic acid molecule can be double-stranded or single-stranded (i.e., a sense or an antisense single strand). Nucleic acids include, for example, cDNAs encoding the light and heavy chains of the B7-DC XAb antibody.

An “isolated nucleic acid” refers to a nucleic acid that is separated from other nucleic acid molecules that normally flank one or both sides of the nucleic acid in the genome in which it is normally found. The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in its naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid.

The isolated nucleic acids disclosed herein can encode polypeptides provided herein. For example, an isolated nucleic acid can encode a polypeptide containing an amino acid sequence that is similar or identical to an amino acid sequence found in the variable or constant regions of B7-DC XAb. In one embodiment, a nucleic acid can encode a polypeptide containing an amino acid sequence that is at least 80.0% identical (e.g., 80.0%, 85.0%, 90.0%, 95.0%, 95.5%, 96.0%, 96.5%, 97.0%, 97.5%, 98.0%, 98.5%, 99.0%, 99.1%, 99.2%, 99.3%, 99.4%, 99.5%, 99.6%, 99.7%, 99.8%, 99.9%, from 80.0% to 99.9%, from 95.0% to 99.9%, from 96.0% to 99.9%, from 97.0% to 99.9%, or from 98.0% to 99.9% identical) to the amino acid sequence set forth in SEQ ID NO:3 or SEQ ID NO:5. The encoded polypeptide can further contain an amino acid sequence that is at least 80.0% identical (e.g., 80.0%, 85.0%, 90.0%, 95.0%, 95.5%, 96.0%, 96.5%, 97.0%, 97.5%, 98.0%, 98.5%, 99.0%, 99.1%, 99.2%, 99.3%, 99.4%, 99.5%, 99.6%, 99.7%, 99.8%, 99.9%, from 80.0% to 99.9%, from 95.0% to 99.9%, from 96.0% to 99.9%, from 97.0% to 99.9%, or from 98.0% to 99.9% identical) to the amino acid sequence set forth in SEQ ID NO:4, SEQ ID NO:6, SEQ ID NO:7, SEQ ID NO:8, or SEQ ID NO:9. In another embodiment, an isolated nucleic acid can contain a nucleotide sequence that is at least 80.0% identical (e.g., 80.0%, 85.0%, 90.0%, 95.0%, 95.5%, 96.0%, 96.5%, 97.0%, 97.5%, 98.0%, 98.5%, 99.0%, 99.1%, 99.2%, 99.3%, 99.4%, 99.5%, 99.6%, 99.7%, 99.8%, 99.9%, from 80.0% to 99.9%, from 95.0% to 99.9%, from 96.0% to 99.9%, from 97.0% to 99.9%, or from 98.0% to 99.9% identical) to the nucleotide sequence set forth in SEQ ID NO:10 or SEQ ID NO:11. The method for determining percent sequence identity is provided herein.

The isolated nucleic acid molecules provided herein can be produced by standard techniques, including, without limitation, common molecular cloning and chemical nucleic acid synthesis techniques. For example, polymerase chain reaction (PCR) techniques can be used to obtain an isolated nucleic acid molecule encoding an antibody such as B7-DC XAb. Isolated nucleic acids provided herein also can be chemically synthesized, either as a single nucleic acid molecule (e.g., using automated DNA synthesis in the 3′ to 5′ direction using phosphoramidite technology) or as a series of polynucleotides. For example, one or more pairs of long polynucleotides (e.g., >100 nucleotides) can be synthesized that contain the desired sequence, with each pair containing a short segment of complementarity (e.g., about 15 nucleotides) such that a duplex is formed when the polynucleotide pair is annealed. DNA polymerase is used to extend the polynucleotides, resulting in a single, double-stranded nucleic acid molecule per polynucleotide pair.

This document also provides vectors containing nucleic acids such as those described above. As used herein, a “vector” is a replicon, such as a plasmid, phage, or cosmid, into which another DNA segment may be inserted so as to bring about the replication of the inserted segment. The vectors provided herein can be expression vectors. An “expression vector” is a vector that includes one or more expression control sequences, and an “expression control sequence” is a DNA sequence that controls and regulates the transcription and/or translation of another DNA sequence.

In the expression vectors provided herein, a nucleic acid (e.g., a nucleic acid encoding the light and/or heavy chains of B7-DC XAb) is operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 to 500 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence. Expression vectors provided herein thus are useful to produce B7-DC XAb, as well as other molecules provided herein.

Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

An expression vector can include a tag sequence designed to facilitate subsequent manipulation of the expressed nucleic acid sequence (e.g., purification or localization). Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide including at either the carboxyl or amino terminus.

This document also provides host cells containing vectors provided herein. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Suitable methods for transforming and transfecting host cells are found in Sambrook et al., Molecular Cloning: A Laboratory Manual (2^{nd} edition), Cold Spring Harbor Laboratory, New York (1989), and reagents for transformation and/or transfection are commercially available (e.g., Lipofectin® (Invitrogen/Life Technologies); Fugene (Roche, Indianapolis, Ind.); and SuperFect (Qiagen, Valencia, Calif.)).

### 4. Compositions

The molecules described herein (e.g., antibodies such as B7-DC XAb and polypeptides such as PD-1) can be incorporated into compositions. Such compositions are provided herein, as is the use of B7-DC binding molecules in the manufacture of compositions. The compositions provided herein can be administered to a subject in order to enhance DC function and potentiate an immune response. Such compositions also can be useful to inhibit Th2 immune responses, and thus can treat or inhibit development of allergic asthma. As described herein, enhanced DC function includes such components as prolonged longevity, increased ability to activate naïve T-cells, increased localization to the lymph nodes, increased phosphorylation of AKT, and increased secretion of interleukin-12 (IL-12).

Compositions provided herein also can contain a molecule (e.g., PD-1) that is immobilized on a solid substrate. Such compositions can be used to contact DC and enhance their function as described above.

Methods for formulating and subsequently administering therapeutic compositions are well known to those skilled in the art. Dosages typically are dependent on the responsiveness of the subject to the molecule, with the course of treatment lasting from several days to several months, or until a suitable immune response is achieved. Persons of ordinary skill in the art routinely determine optimum dosages, dosing methodologies and repetition rates. Optimum dosages can vary depending on the relative potency of an antibody, and generally can be estimated based on the EC₅₀ found to be effective in in vitro and/or in vivo animal models. Dosage typically is from 0.01 μg to 100 g per kg of body weight (e.g., from 1 μg to 100 mg, from 10 μg to 10 mg, or from 50 μg to 500 μg per kg of body weight). Compositions containing the molecules provided herein may be given once or more daily, weekly, monthly, or even less often.

In addition to the molecules provided herein, the compositions described herein further can contain antigens that will elicit a specific immune response. Suitable antigens include, for example, polypeptides or fragments of polypeptides expressed by tumors and pathogenic organisms. Killed viruses and bacteria, in addition to components of killed viruses and bacteria, also are useful antigens. Such antigens can stimulate immune responses against tumors or pathogens.

Compositions also can include DC that have been isolated from, for example, bone marrow, spleen, or thymus tissue. DC lines also can be useful in compositions provided herein. The DC can be activated by a molecule as described herein, and also can be contacted by one or more antibodies against the T-cell receptor complex. In some embodiments, for example, a B7-DC XAb-activated DC can be coated on its Fc receptors with an antibody that specifically binds to CD3 E. Other suitable components of the T-cell receptor complex against which an antibody can be directed as described herein include, without limitation, the T-cell receptor α or β chain, as well as CD3α, CD3β, CD3δ, and CD3ζ. In some embodiments, a bifunctional antibody can be prepared, such that it is targeted to a component of the T-cell receptor complex and is attached to any suitable cell surface molecule.

The molecules featured herein (e.g., antibodies such as B7-DC XAb) can be admixed, encapsulated, conjugated or otherwise associated with other molecules, molecular structures, or mixtures of compounds such as, for example, liposomes, receptor targeted molecules, or oral, topical or other formulations for assisting in uptake, distribution and/or absorption.

In some embodiments, a composition can contain a molecule provided herein (e.g., B7-DC XAb, a polypeptide containing an amino acid sequence that is at least 80.0% identical to SEQ ID NO:3 or SEQ ID NO:5, a nucleic acid encoding a polypeptide that contains an amino acid sequence at least 80.0% identical to the sequence set forth in SEQ ID NO:3 or SEQ ID NO:5, or a nucleic acid molecule containing a nucleotide sequence that is at least 80.0% identical to the sequence set forth in SEQ ID NO:10 or SEQ ID NO:11) in combination with a pharmaceutically acceptable carrier. Activated DC (e.g., DC activated by B7-DC XAb and contacted with an antibody against the T-cell receptor complex) also can be combined with a pharmaceutically acceptable carrier. Pharmaceutically acceptable carriers are pharmaceutically acceptable solvents, suspending agents, or any other pharmacologically inert vehicles for delivering antibodies to a subject. Pharmaceutically acceptable carriers can be liquid or solid, and can be selected with the planned manner of administration in mind so as to provide for the desired bulk, consistency, and other pertinent transport and chemical properties, when combined with one or more therapeutic compounds and any other components of a given pharmaceutical composition. Typical pharmaceutically acceptable carriers include, without limitation: water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

Pharmaceutical compositions containing molecules provided herein can be administered by a number of methods, depending upon whether local or systemic treatment is desired. Administration can be, for example, parenteral (e.g., by subcutaneous, intrathecal, intraventricular, intramuscular, or intraperitoneal injection, or by intravenous (i.v.) drip); oral; topical (e.g., transdermal, sublingual, ophthalmic, or intranasal); or pulmonary (e.g., by inhalation or insufflation of powders or aerosols). Administration can be rapid (e.g., by injection) or can occur over a period of time (e.g., by slow infusion or administration of slow release formulations). For administration to the central nervous system, antibodies can be injected or infused into the cerebrospinal fluid, typically with one or more agents capable of promoting penetration across the blood-brain barrier.

Compositions and formulations for parenteral, intrathecal or intraventricular administration include sterile aqueous solutions (e.g., sterile physiological saline), which also can contain buffers, diluents and other suitable additives (e.g., penetration enhancers, carrier compounds and other pharmaceutically acceptable carriers).

Compositions and formulations for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders.

Formulations for topical administration include, for example, sterile and non-sterile aqueous solutions, non-aqueous solutions in common solvents such as alcohols, or solutions in liquid or solid oil bases. Such solutions also can contain buffers, diluents and other suitable additives. Pharmaceutical compositions and formulations for topical administration can include transdermal patches, ointments, lotions, creams, gels, drops, suppositories, sprays, liquids, and powders. Conventional pharmaceutical carriers, aqueous, powder or oily bases, thickeners and the like may be useful.

Pharmaceutical compositions include, but are not limited to, solutions, emulsions, aqueous suspensions, and liposome-containing formulations. These compositions can be generated from a variety of components that include, for example, preformed liquids, self-emulsifying solids and self-emulsifying semisolids. Emulsion formulations are particularly useful for oral delivery of therapeutic compositions due to their ease of formulation and efficacy of solubilization, absorption, and bioavailability. Liposomes can be particularly useful due to their specificity and the duration of action they offer from the standpoint of drug delivery.

Molecules featured herein can encompass any pharmaceutically acceptable salts, esters, or salts of such esters, or any other compound which, upon administration to a subject, is capable of providing (directly or indirectly) the biologically active metabolite or residue thereof. Accordingly, for example, this document provides pharmaceutically acceptable salts of molecules such as antibodies (e.g., B7-DC XAb), prodrugs and pharmaceutically acceptable salts of such prodrugs, and other bioequivalents. A prodrug is a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of the antibodies useful in methods described herein (i.e., salts that retain the desired biological activity of the parent antibodies without imparting undesired toxicological effects). Examples of pharmaceutically acceptable salts include, but are not limited to, salts formed with cations (e.g., sodium, potassium, calcium, or polyamines such as spermine); acid addition salts formed with inorganic acids (e.g., hydrochloric acid, hydrobromic acid, sulfuric acid, phosphoric acid, or nitric acid); salts formed with organic acids (e.g., acetic acid, citric acid, oxalic acid, palmitic acid, or fumaric acid); and salts formed with elemental anions (e.g., bromine, iodine, or chlorine).

Compositions additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions provided herein, such as dyes, flavoring agents, preservatives, antioxidants, opacifiers, thickening agents, and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, penetration enhancers, and aromatic substances. When added, however, such materials should not unduly interfere with the biological activities of the PNA components within the compositions provided herein.

Pharmaceutical formulations as disclosed herein, which can be presented conveniently in unit dosage form, can be prepared according to conventional techniques well known in the pharmaceutical industry. Such techniques include the step of bringing into association the active ingredients (i.e., the antibodies) with the desired pharmaceutical carrier(s). Typically, the formulations can be prepared by uniformly and intimately bringing the active ingredients into association with liquid carriers or finely divided solid carriers or both, and then, if necessary, shaping the product. Formulations can be sterilized if desired, provided that the method of sterilization does not interfere with the effectiveness of the antibody(s) contained in the formulation.

Compositions can be formulated into any of many possible dosage forms such as, without limitation, tablets, capsules, liquid syrups, soft gels, suppositories, and enemas. Compositions also can be formulated as suspensions in aqueous, non-aqueous or mixed media. Aqueous suspensions further can contain substances that increase the viscosity of the suspension including, for example, sodium carboxymethyl-cellulose, sorbitol, and/or dextran. Suspensions also can contain stabilizers.

### 5. Methods

This document provides methods for potentiating an immune response in a mammal, modulating a state of immune response (e.g., altering the polarity of an immune response, or altering the activity of regulatory T cells (Treg)) in a mammal, inhibiting a Th2 immune response in a mammal, and inhibiting (e.g., reducing or preventing) tumor growth in a mammal.

The methods provided herein typically include administering to a mammal (e.g., a dog, a cat, a horse, a cow, a rabbit, a rat, a mouse, or a human) a molecule (e.g., an antibody such as B7-DC XAb) or a composition described herein (e.g., a composition containing an antibody such as B7-DC XAb, with or without an antigen). Methods also can involve administration of DC that have been contacted with a molecule or a composition provided herein. For example, DC can be contacted with B7-DC XAb, with a composition containing B7-DC XAb and an antigen, or with B7-DC XAb and an antibody specific for a component of the T-cell receptor complex. Such molecules, compositions, and DC can be useful to, for example, potentiate an immune response or alter the polarity of an immune response in the mammal to which they are administered, or to modulate Treg function from a suppressor phenotype to an effector phenotype. B7-DC XAb-activated DC contacted with an antibody against a component of the T-cell receptor complex (e.g., coated with an anti-CD3 antibody bound to the DC Fc receptor) also can be useful to inhibit tumor growth in a mammal having or at risk for having a tumor.

As described above, the molecule, composition, or activated DC can be administered by any suitable systemic or local method. Systemic methods of administration include, without limitation, oral, topical, or parenteral administration, as well as administration by injection. Local methods of administration include, for example, direct injection into a tumor.

Methods provided herein also can be used to modulate (e.g., enhance) DC function. The enhancement of DC function includes, for example, prolonging the longevity of DC, increasing the ability of DC to activate naïve T-cells, and increasing the localization of DC to lymph nodes in a mammal. The longevity of DC can be assessed by, for example, measuring the expression of NF-κB or the translocation of NF-κB to the nucleus. Since NF-κB is an intracellular signal involved in the inhibition of programmed cell death, increased expression or translocation of NF-κB indicates inhibition of apoptosis and prolonged DC longevity. T-cell activation can be measured by, for example, assessing the incorporation of radiolabeled (e.g., tritiated) thymidine into newly synthesized DNA in proliferating T-cells. Activation of naïve T-cells also can be measured by detecting (e.g., by flow cytometry) CD44 and/or CD69 activation markers on the T-cell surface.

### 6. Articles of Manufacture

This document provides articles of manufacture that can include one or more molecules and/or compositions disclosed herein. The molecule and/or composition can be combined with packaging material and sold as kits for altering immune responses. The molecule and/or composition can be in a container such as a vial, a tube, or a syringe, for example, and can be at least partially surrounded with packaging material. Components and methods for producing articles of manufacture are well known.

Articles of manufacture may combine one or more of the molecules set out in the above sections. For example, an article of manufacture can contain a composition that includes a molecule provided herein (e.g., an antibody such as B7-DC XAb or a polypeptide such as immobilized PD-1). An article of manufacture also can include one or more antigens (e.g., a tumor antigen or an antigen from a pathogen) that can stimulate a specific immune response. Furthermore, an article of manufacture can contain DC. The DC can be activated (e.g., with B7-DC XAb), and can be contacted with an antibody specific for a component of the T-cell receptor complex (e.g., an anti-CD3 antibody). An article of manufacture also may include, for example, buffers or other control reagents for potentiating an immune response. Instructions indicating that the molecules, antigens, DC, and/or compositions are effective for potentiating an immune response or for treating or reducing development of allergic asthma also can be included in such kits.

The invention will be further described in the following examples, which do not limit the scope of the invention described in the claims.

### Examples

### Example 1

### Isolation of B7-DC XAb

Human serum samples were obtained from the dysproteinemia clinic, and those exhibiting an Ig clonal peak of greater than 20 mg/ml were chosen for further evaluation. The selected samples were from 50 patients with a wide variety of conditions characterized by a monoclonal IgM spike, including Waldenstrom's macroglobulinemia, lymphoma, and monoclonal gammopathy of undetermined significance. Sera were dialyzed against water, and precipitates were collected by centrifugation at 14,000 rpm for 30 minutes and dissolved in phosphate buffered saline (PBS). The samples were centrifuged and subjected to chromatography on a Superose-6 column (Amersham Pharmacia, Piscataway, N.J.). IgM fractions were pooled and analyzed by SDS-PAGE, and protein concentrations were determined by reading absorbance at 280 nm. IgM solutions were sterile filtered and cryopreserved. The antibody B7-DC XAb (also referred to as sHIgM12 or Lym 12) was identified based on its ability to bind DC as determined by FACS analysis. The polyclonal human IgM antibody control was described previously (Miller et al. (1994) J. Neurosci. 14:6230-6238).

A vector system was developed to generate an immortalized source of B7-DC XAb. An amino acid sequence for B7-DC XAb was obtained. Since the amino-terminus of the antibody heavy chain was blocked, Fv fragments were generated from serum to increase the efficiency of obtaining an amino terminal sequence. The amino terminal sequence of the B7-DC XAb heavy chain was determined to be Val-Gln-Leu-Gln-Glu-Ser-Gly-Pro-Gly-Leu-Leu-Lys-Pro-Ser-Glu-Thr-Leu-Arg/Ser-Leu-Thr-Asn (SEQ ID NO:1), while the amino terminal sequence of the light chain was determined to be Asp-Ile-Gln-Met-Thr-Gln-Ser-Pro-Ser-Ser-Leu-Ser-Ala-Ser-Val-Gly-Asp-Arg-Val (SEQ ID NO:2).

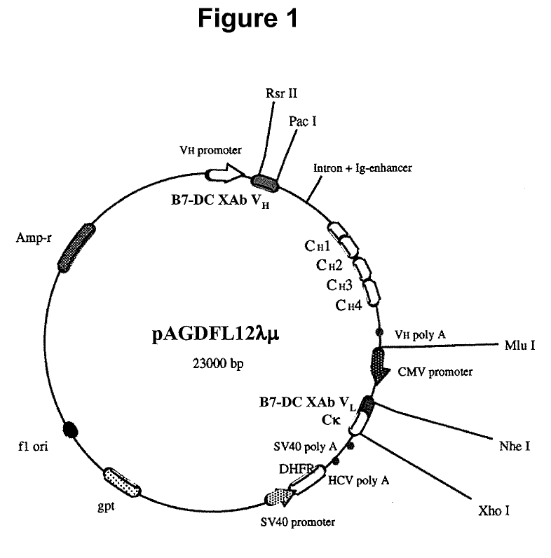

cDNA was isolated from the patient's peripheral blood cells, to be used for recovering full length cDNA copies of the mRNA encoding B7-DC XAb. In order to ensure than recovered cDNAs truly represented the antibody of interest, the amino acid sequences of CDR3 regions of B7-DC XAb were determined. This was accomplished by proteolytic digestion of the Fv fragments and conventional amino acid sequencing of the digestion products. Once the B7-DC XAb cDNAs were obtained, they were used to generate a genomic human IgM heavy chain gene encoding the variable region derived from the patient antibody and a cDNA-based light chain gene expressed under control of a cytomegalovirus (CMV) promoter. These antibody gene sequences were incorporated into a single vector (FIG. 1) along with a selectable dHfR gene expressed under the control of a SV40 promoter. The vector bearing the synthetic antibody genes was introduced into human/mouse F3B6 hybridoma cells by electroporation. Methotrexate resistant cells were selected and amplified by stepping up the amount of methotrexate in the culture medium. A clone expressing 100 μg antibody per ml of supernatant was recovered. The recombinant antibody displayed all functional properties identified for the antibody isolated from the patient serum.

Amino acid sequences for the variable (Vk) and constant (Ck) domains of the B7-DC XAb light chain are set forth in SEQ ID NOS:3 and 4, respectively. Amino acid sequences for the variable (Vh) and constant (CH1, CH2, CH3, and CH4) domains of the B7-DC XAb heavy chain are set forth, respectively, in SEQ ID NOS:5, 6, 7, 8, and 9. Nucleotide sequences encoding the variable regions of the B7-DC XAb light and heavy chains are shown in SEQ ID NOS:10 and 11, respectively. The recombinant antibody was deposited in the American Type Culture Collection (ATCC) on Aug. 24, 2004, and was assigned Patent Deposit Designation number PTA-6164.

### Example 2

### B7-DC XAb Induces Phenotype Reversal in an Antigen Driven Recall Response from Th2 to Th1 Polarity

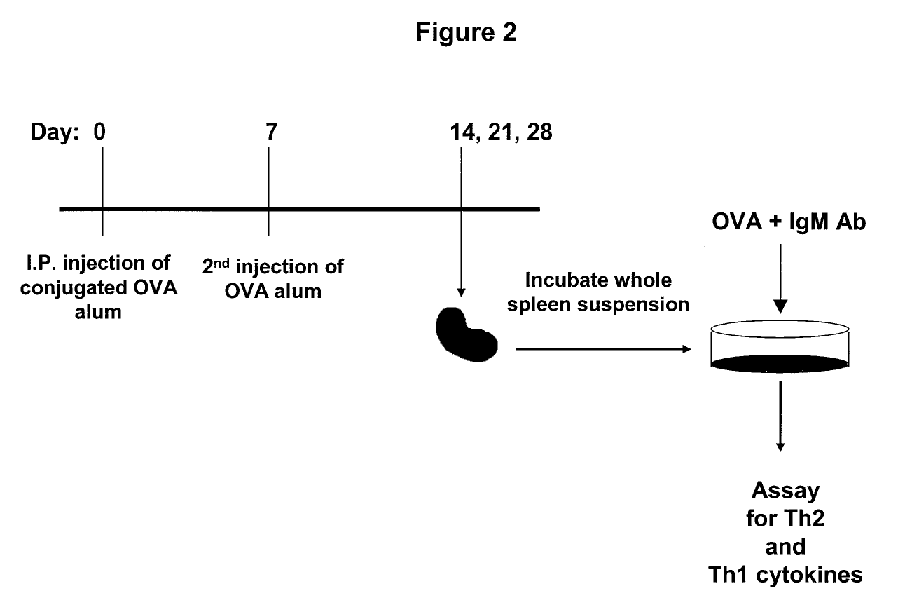

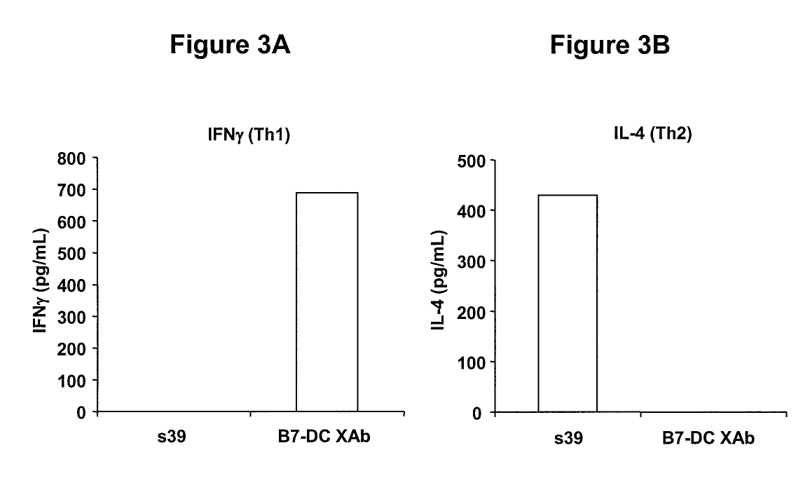

As depicted in FIG. 2, BALB/c mice were immunized on days zero and 7 by intraperitoneal (i.p.) injection with 100 μg of the chicken ovalbumin (OVA) antigen (Ag), adsorbed to an alum adjuvant. On day 14, 21, or 28 after the first immunization, spleen cells were removed and placed in culture with OVA and either 10 μg/ml of the B7-DC XAb IgM antibody or the s39 isotype control IgM antibody. Two days later, culture supernatants were collected and assayed for the presence of the cytokines IL-4 (a marker for the Th2 response phenotype) or IFNγ (a marker for the Th1 response phenotype). As shown in FIG. 3, B7-DC XAb caused a switch in the polarity of the immune response, from a Th2 response to a Th1 response.

### Example 3

### B7-DC Activated DC Coated with Anti-CD3 Antibodies can Modulate the Memory Response to a Recall Antigen

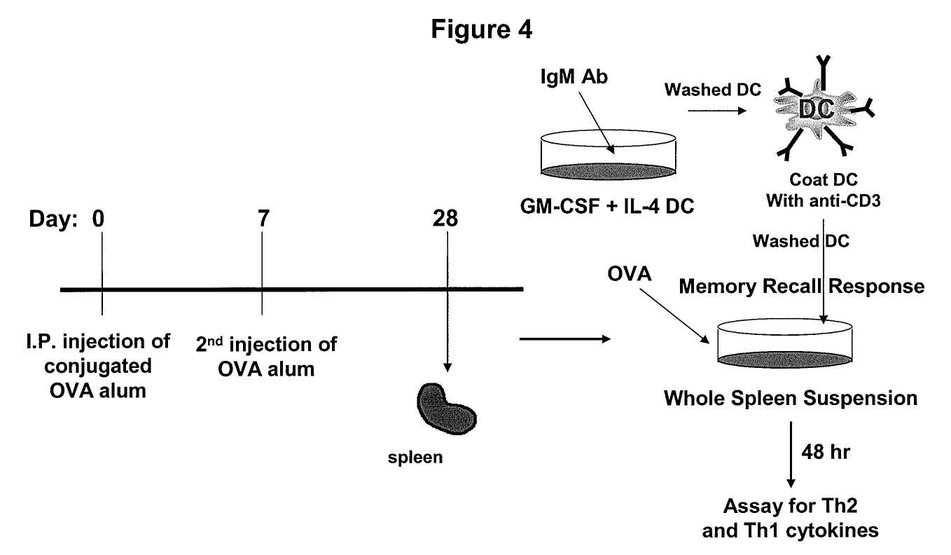

Experiments were conducted as described in Example 2, except instead of adding IgM antibody directly to spleen cell cultures to assess polarity of the memory response, six day bone marrow derived mouse DC were treated in a separate Ag-free culture with B7-DC XAb or s39 isotype control IgM antibody (FIG. 4). Activated DC were then washed and coated with Armenian hamster anti-mouse CD3 antibody. The antibody coated, activated DC were used to modulate the immune response of primed T-cells to re-exposure to the OVA inciting Ag (addition of recall OVA Ag to the final spleen cell cultures was required to drive potent Th2 or Th1 responses in two days). After 48 hours, supernatants were analyzed for levels of IL-4 and IFNγ.

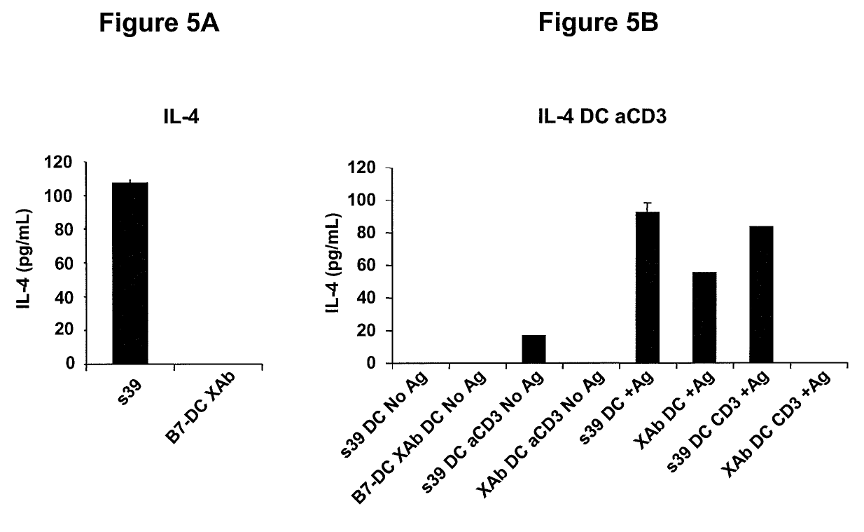

FIG. 5 includes data from experiments evaluating Th2 recall responses by spleen cells supplemented with exogenous DC. In FIG. 5A, the ability of the memory spleen cell population to undergo B7-DC XAb-induced modulation while responding to a recall Ag was documented by adding IgM antibodies directly to spleen cell cultures as depicted in FIG. 2. In FIG. 5B, DC were added to spleen cell cultures along with the recall OVA Ag as depicted in FIG. 4. The treatment groups tested were DC treated with isotype control antibody (s39), DC treated with B7-DC XAb (XAb), and DC treated with isotype control antibody or B7-DC XAb and coated with anti-CD3 antibody. In the first four groups, no Ag was added to the spleen cell culture in order to assess whether the DC were sufficient to drive a response. A minimal amount of the Th2 cytokine IL-4 was detected only in the culture that received DC treated with the s39 isotype control IgM antibody and coated with anti-CD3 antibody. The second set of four cultures was a duplicate of the first set, except that Ag was added to primed spleen cells along with the DC. In these cases, a strong Th2 response (IL-4) was detected in the cultures receiving s39 isotype control IgM antibody treated DC, B7-DC XAb-treated DC, and isotype control IgM antibody-treated/anti-CD3-coated DC. No IL-4 was detected, however, in the culture that received B7-DC XAb-activated DC coated with anti-CD3 antibodies. These results demonstrate that antigen must be present at the time of dendritic cell activation with B7-DC XAb or, alternatively, the activated DC must be armed with the MHC/Ag surrogate, anti-CD3 antibody, in order to modulate the recall response of primed spleen cells.

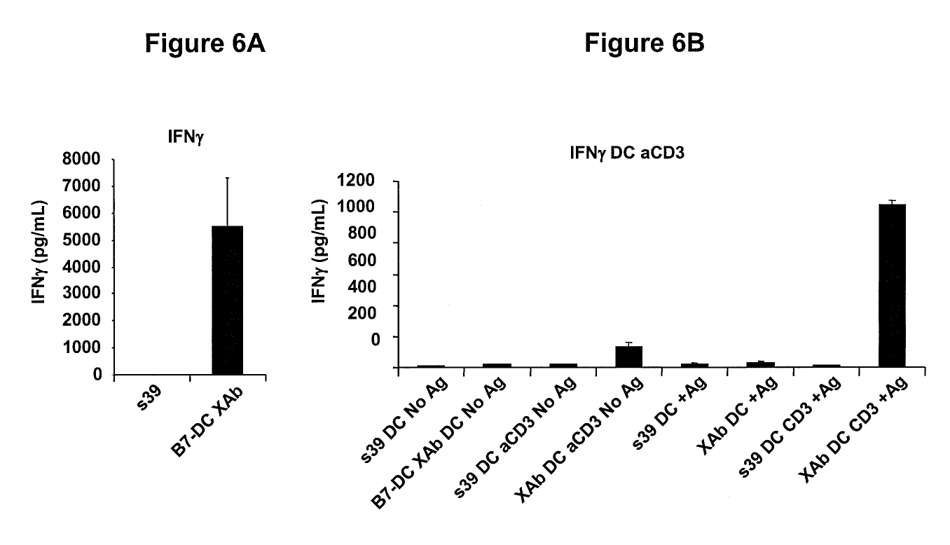

FIG. 6 includes data from experiments evaluating Th1 recall responses by spleen cells supplemented with exogenous DC. Supernatants collected in the experiment depicted in FIG. 4 were analyzed for the presence of the IFNγ as a marker for a Th1 polarized response. In FIG. 6A, IFNγ was detected in the supernatants of cultures treated with B7-DC XAb and Ag. In FIG. 6B, a weak IFNγ response by primed spleen cells was induced by B7-DC XAb-activated, anti-CD3 coated DC in the absence of recall Ag, indicating modulation of polarity, even in the absence of Ag. A strong Th1 response was clearly seen in cultures that received both B7-DC XAb-activated, anti-CD3 coated DC and the recall Ag OVA. However, B7-DC XAb-activated DC could not modulate the recall response even when Ag and the DC were added to the spleen cultures at the same time, demonstrating that the activated DC could not acquire Ag from the spleen cell culture and that direct recognition of the activated DC is required to modulate the immune response in these cultures.

Taken together, these experiments showed that DC treated with s39 isotype control human IgM antibody did not alter the Th2 response to OVA, whether or not they were coated with anti-CD3 antibody. In contrast, DC activated with B7-DC XAb and coated with Ag modulated the response of the primed spleen cells to OVA Ag, resulting in loss of IL-4 production and gain of IFNγ secretion.

### Example 4

### Modulation of the Polarity of the Immune Response Persists in the Absence of Antigen

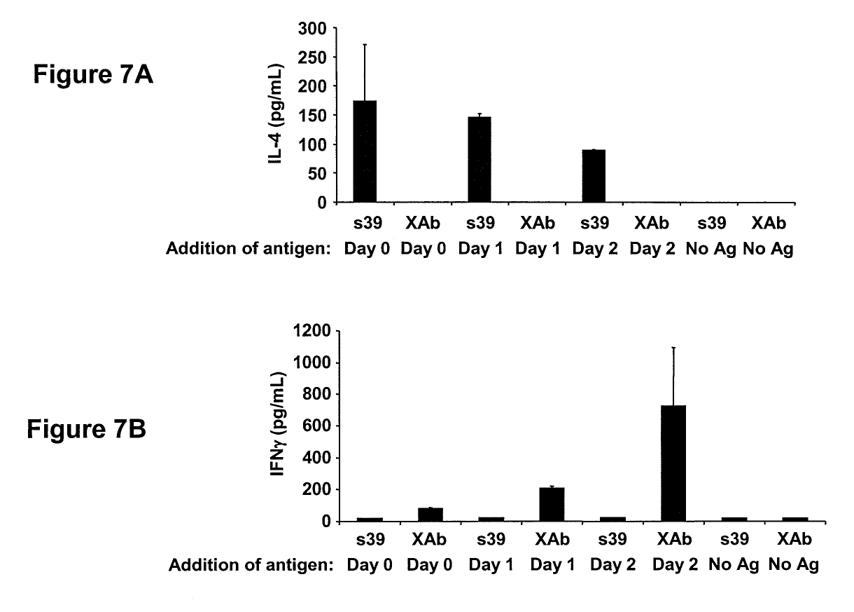

Experiments were conducted to assess whether modulation of the immune response required the presence of Ag added at the time of first exposure of primed spleen cells to CD3-coated DC. These experiments were similar to those described in Example 2, except that Ag was added to the cultures either at the same time as IgM antibody treated DC (time 0), 1 or 2 days after addition of DC, or not at all. Culture supernatants were assayed for the presence of IL-4 (Th2) and IFNγ (Th1) two days after addition of Ag. As shown in FIG. 7A, B7-DC XAb-activated DC (XAb) that were coated with anti-CD3 antibody inhibited the Th2 recall response, irrespective of when Ag was added to the culture. Conversely, as shown in FIG. 7B, XAb-activated DC coated with anti-CD3 antibody induced a Th1 recall response, irrespective of when Ag was added to the culture. Remarkably, the data suggest that the Th1 recall response improved over time, raising the possibility that the DC were amplifying the recall response.

### Example 5

### Anti-Tumor Protection Induced In Vivo using B7-DC XAb-Activated DC Coated with Anti-CD3 Antibody

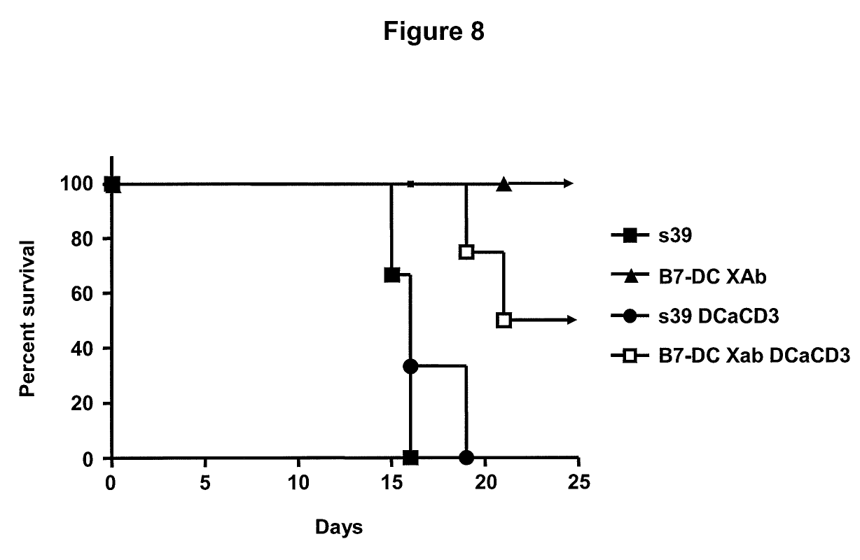

Studies were conducted to evaluate whether B7-DC XAb-activated, anti-CD3 coated DC can modulate an immune response in vivo. Four groups of six C57BL/6 mice were challenged with a subcutaneous lethal inoculum of 5×10⁵ B16 melanoma cells. The animals also received systemic treatment with 10 μg/100 μl B7-DC XAb, s39 isotype control antibody, or 1 million CD3-coated DC that had been activated in vitro overnight with either B7-DC XAb or s39. Treatments were administered intravenously in PBS on days −1, 0, and +1 relative to the tumor challenge. Tumor growth was monitored over 100 days, and the percentage of animals that were tumor free was for each treatment group was recorded. All six animals treated with the B7-DC XAb-activated, anti-CD3-coated DC remained tumor free for a longer period than did the six mice that received control antibody treated, anti-CD3 coated DC (P<0.05). Half the mice treated with the protective protocol never developed melanoma throughout the duration of the observation period. As shown in FIG. 8, all animals that received the control antibody, with or without the activated DC, were dead by day 20. In contrast, all animals that received B7-DC XAb survived at least to day 25. These experiments demonstrated that B7-DC XAb-activated DC coated with anti-CD3 antibody can induce a protective immune response in vivo even though the Ag presenting cells have not been pulsed with the relevant tumor antigens.

### Example 6

### Materials and Methods for Examples 7-13

Mice and reagents: C57BL/6J, BALB/CJ, B6.129S4-Cd80^{tm1Shr}Cd86^{tm1Shr/J} (Borriello et al. (1997) Immunity 6:303-313), B6.129S4-Icam1^{tm1Jcgr/J} (Xu et al. (1994) J. Exp. Med. 180:95-109), and C.129s1(B6)-Il12a^{tm1Jm/J} (Mattner et al. (1996) Eur. J. Immunol. 26:1553-1559) mice were purchased from The Jackson Laboratory (Bar Harbor, Me.). Signaling lymphocytic activation molecule (SLAM)^{-/-} (Wang et al. (2004) J. Exp. Med. 199:1255-1264) mice were maintained at the Mayo Clinic (Rochester, Minn.). LY9^{-/-} (Graham et al. (2006) J. Immunol. 176:291-300) mice and CD4⁺ Stat4^{-/-}tm^{gru/J} (Kaplan et al. (1996) Nature 382:174-177), Stat6^{-/-}tmg^{gru/J} (Kaplan (1996) Immunity 4:313-319), C57BL/6-IFN-γ^{tm1Ts} (Wang et al. (1994) J. Exp. Med. 179:1367-1371), and 129-IFN-γR^{tm1} (Huang et al. (1993) Science 259:1742-1745) mice, along with their appropriate haplotype-matched controls, were obtained from Dr. M. Rodriguez, Mayo Clinic.

The clonal CD4⁻ T cell line clone 10 was previously described (McKean et al. (1985) J. Immunol. 135:3205-3216). Control Ab and B7-DC XAb were purified as described (above; and Radhakrishnan et al. (2003) J. Immunol. 170:1830-1838). The class II IgM Ab (25-9-3), antibodies against mouse IFN-γ-FITC (clone XMG1.2), IL-4-PE (clone 11B11) CD3-PerCP (clone 145-2C11), and CD4-PerCP (RM4-5) were purchased from BD Biosciences (San Jose, Calif.). An antibody against CD40 (clone 1C10) and an antibody against mouse B7-DC of IgG isotype (clone TY-25) were purchased from eBioscience. CpG (TCCATGACGTTCCTGACGTT; SEQ ID NO:12) was synthesized at the Mayo Clinic Molecular Biology Core Facility (Rochester, Minn.). Polyinosinic:polycytidylic acid (pI:C) was purchased from Calbiochem (San Diego, Calif.). Gardiquimod was purchased from InvivoGen (San Diego, Calif.). LPS, OVA, and BSA were purchased from Sigma-Aldrich (St. Louis, Mo.). CpG, LPS, pI:C, and Gardiquimod were frozen at −20° C. as 1 mg/ml stocks. T cell enrichment kits were purchased from R&D Systems (Minneapolis, Minn.). Cytokine ELISA kits for measuring IL-4, IL-5, and IFN-γ were purchased from eBioscience (San Diego, Calif.).

Multiplexed microsphere cytokine immunoassay: A Bio-Rad mouse cytokine panel was used for this study. The kit measures concentrations of IL-3, IL-4, IL-5, IL-6, IL-8, IL-10, IL-12(p70), GM-CSF, M-CSF, IL-17, IFN-γ, and RANTES. The assay was performed as per the manufacturer's direction along with standards. Briefly, 100 ml of Bio-Plex assay buffer were added to each well of a MultiScreen MABVN 1.2-μm microfiltration plate, followed by the addition of 50 μl of the multiplex bead preparation. After washing of the beads with the addition of 100 μl of wash buffer, 50 μl of the samples or standards were added to each well and incubated with shaking for 30 minutes at room temperature. The plate was then washed three times, followed by incubation of each well in 25 μl of premixed detection antibodies for 30 minutes with shaking. The plate was further washed, and 50 μl of streptavidin solution was added to each well and incubated for 10 minutes at room temperature with shaking. The beads were given a final washing and resuspended in 125 μl of Bio-Plex assay buffer. Cytokine levels in the culture supernatant were quantitated by analyzing 100 μl of each sample on a Bio-Plex using Bio-Plex Manager software, version 4.0. Standard curves were generated with a mixture of cytokine standards provided by the supplier and eight serial dilutions ranging from 0 to 32,000 pg/ml.

Generation of mouse DC: DC from mouse bone marrow was generated as previously described (Radhakrishnan et al. supra). In brief, bone marrow was isolated from the long bones of the hind legs. Erythrocytes were lysed by treatment with ammonium chloride/potassium bicarbonate/EDTA at 37° C. The remaining cells were plated at the density of 1×10⁶/ml in six-well plates (BD Biosciences) in RPMI 1640 containing 10 ng/ml murine GM-CSF and 1 ng/ml murine IL-4 (PeproTech; Rocky Hill, N.J.). The cells were incubated at 37° C. with 5% CO₂. After 48 hours, the cells were washed and replated with RPMI 1640 containing the same concentration of GM-CSF and IL-4 for another 5 days.

Induction of Th2 polarity: Mice were immunized by an i.p. injection of 1 mg of OVA or BSA adsorbed to 1 mg of alum (Pierce; Rockford, Ill.). Experimental mice were later sacrificed, and splenocytes were harvested as early as 2 weeks or as late as 8 weeks after injection and used to generate Ag-specific recall responses.

In vitro cytokine production and proliferation: Recall responses from sensitized splenocytes were assayed for cytokine production or proliferation. Briefly, after making a single cell suspension, red blood cells (RBC) were lysed by hypertonic shock using ammonium chloride/potassium bicarbonate/EDTA. Cells were counted and resuspended at 3 million cells/ml in RPMI 1640 (Cambrex; East Rutherford, N.J.) along with an antigen and a panel of activators. OVA and BSA were used as antigens, prepared at a concentration of 2 mg/ml, and titrated at half-log dilutions. Splenocytes were used at 3×10⁵ cells per well in 100 μl. DC, used as stimulators, were pulsed with 1 mg/ml OVA, BSA, or sperm whale myoglobulin, and treated with the indicated activators overnight at 10 μg/ml. The DC were washed extensively before being used to stimulate Th2-polarized T cells or the T cell line clone 10. Supernatants were harvested after 48 hours and stored frozen at −20° C. until used in ELISA-based cytokine assays as per the manufacturer's instructions using commercial kits. The plates were read using a SoftPro plate reader. In some experiments, cells were pulsed with [³H]thymidine during the last 18 hours of the 72-hour incubation period. Cells were then harvested and counted for the incorporation of [³H]thymidine (Packard Instrument; Meriden, Conn.).

DC and T cell enrichment: CD11c⁺ DC were enriched from cultures by positive selection using magnetic bead sorting (Miltenyi Biotec; Bergisch Gladbach, Germany). CD11c⁺ DC enrichment varied from 70 to 85%. The remaining cells were CD11c CD11b^{high}Gr-1^{int}, a phenotype consistent with macrophage-like cells. No CD3⁺ cells were detected in the enriched DC cultures. In T cell enrichment experiments, cell suspensions were fractionated using a mouse T cell enrichment kit as per the manufacturer's instructions (R&D Systems). Briefly, splenocytes were incubated in a T cell enrichment column for 15 minutes, followed by washing and elution with the buffer provided by the manufacturer. The purity of isolated T cells was assessed by flow cytometry as described previously (Radhakrishnan et al., supra). The isolated T cells were >98% CD3⁺.

Intracellular cytokine staining: Splenocytes that were stimulated with Ag-pulsed DC for 48 hours were incubated with 1 μg/ml brefeldin A (GOLGIPLUG™) as per the manufacturer's instructions for 4 hours before being fixed and permeabilized using the Cytofix/Cytoperm kit (BD Pharmingen; San Jose, Calif.). After addition of the IFN-γ FITC antibody and the IL-4 PE antibody, cells were incubated on ice for 30 minutes and subjected to flow cytometry. Data were analyzed using Cell Quest Software (BD Bioscience).

### Example 7

### Polarity of the Recall Response by Sensitized Spleen Cells to Ag Rechallenge can be Modulated In Vitro

Treatment of sensitized mice with the TLR-9 agonist CpG-oligodeoxynucleotide (CpG-ODN) (Parronchi et al. (1999) J. Immunol. 163:5946-5953) or the DC activator B7-DC XAb (Radhakrishnan et al. (2004) J. Immunol. 173:1360-1365; and Radhakrishnan et al. (2005) J. Allergy Clin. Immunol. 116:668-674) modulated the polarity of Ag-specific recall responses and protected mice from experimental inflammatory airway disease. The inventors thus surmised that sensitized T cells can be reprogrammed by interacting with activated DC to mitigate immune-based pathogenesis in models of experimental allergic asthma. To investigate the mechanisms that mediate the polarity modulation of recall responses, events were recapitulated in vitro using spleen cells from mice immunized with chicken OVA adsorbed to the Th2-polarizing adjuvant alum (OVA/alum). Spleen cells isolated 14 or more days after initial immunization respond vigorously to rechallenge with OVA in culture, producing a wide array of cytokines characteristic of Th2 responses (Table 1). When the DC activator B7-DC XAb (10 μg/ml) was added to spleen cell cultures along with OVA, the levels of Th2 cytokines recovered in 48 hours were reduced and, concomitantly, the levels of the Th1 cytokines IFN-γ and IL-12 were increased. This pattern was observed using multiple cultures assessed over a 72-hour period. For most of the subsequent experiments described herein, IL-4 and IFN-γ were selected as prototypic cytokines representing the Th2 and Th1 response patterns, respectively.

TABLE 1
Effects of B7-DC XAb on cytokine levels

| Cytokine | Control Ab | B7-DC XAb | Fold change |
|---|---|---|---|
| IL-4 | 42.6 ± 5 | 21 ± 1 | −2 |
| IL-6 | 487 ± 9 | 152 ± 6 | −3.2 |
| IL-10 | 58 ± 5 | 32 ± 2 | −1.7 |
| GM-CSF | 193 ± 8 | 40 ± 7 | −4.8 |
| IL-3 | 870 ± 8 | 89 ± 0.4 | −9.8 |
| IL-5 | 385 ± 8 | 277 ± 3 | −1.4 |
| M-CSF | 104 ± 3 | 58 ± 0.8 | −1.8 |
| RANTES | 104 ± 3 | 58 ± 0.8 | −1.8 |
| KC (IL-8) | 176 ± 7 | 54 ± 2 | −3.3 |
| IL-17 | 454 ± 9 | 149 ± 5 | −3 |
| IFNγ | 100 ± 11 | 261 ± 2.6 | +2.6 |
| IL-12 p70 | 51 ± 1 | 283 ± 0.4 | +5.5 |

In subsequent experiments, the range of cytokines observed using ELISA to measure cytokine levels was consistently higher than those observed using multiplex-based measurements. The multiplex technology employs commercially prepared reagents and proprietary analytical algorithms. To resolve the basis of the differences in absolute levels of cytokines detected using the two methods, IL-4 and IFN-γ levels were assessed in the same samples used in the multiplex analysis in a subsequent analysis by ELISA, and substantially different estimates of the cytokines were obtained. The ELISA-based estimates were in agreement with the levels measured by ELISA throughout this document. Specifically, in the samples yielding IL-4 estimates of 42.6±5 pg/ml from control Ab-treated spleen cells and 21±1 pg/ml in B7-DC XAb-treated spleen cells by multiplex analysis, levels of IL-4 were determined to be 710±3 pg/ml and 3±1 pg/ml for these same samples by ELISA. Similarly, for IFN-γ levels where the multiplex platform indicated 51±1 pg/ml in control-treated samples, no measurable levels of IFN-γ were detected using ELISA, whereas in supernatants from cultures treated with B7-DC XAb, 994±3 pg/ml IFN-γ was found in comparison to the estimate using the multiplex platform of 283±0.4 pg/ml. Both methods indicated a strong shift in polarity from Th2 to Th1 following treatment of spleen cells with B7-DC XAb. The value of the multiplex system is that it provided insight into the directional change of an array of cytokines, suggesting a global change in the polarity of cytokine production. Nevertheless, it is clear that conclusions derived from these studies, all of which include paired controls in each experiment, should be interpreted cautiously with regard to the absolute quantities of cytokine produced.

### Example 8

### B7-DC XAb is an Efficient Polarity Modulator of the Recall Response by Spleen Cells in Culture

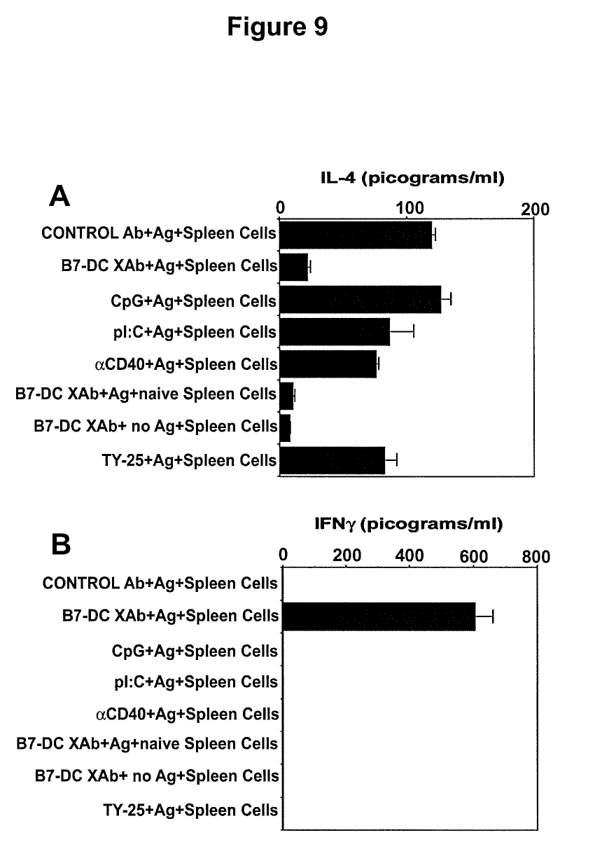

While stimulation of the in vitro recall response against OVA was strongly modulated from Th2 to Th1 by the DC activator B7-DC XAb, the TLR agonists CpG-ODN, pI:C, LPS, and Gardiquomod, and the CD40 agonist anti-CD40 antibody only partially modulated the response. All of these DC activators were added to the cultures at 10 μg/ml. As shown in FIG. 9A, levels of IL-4 were sharply reduced in spleen cell cultures treated with B7-DC XAb, whereas TLR and CD40 agonists induced moderate to no reductions. None of the cultures that received TLR or CD40 agonists produced IFN-γ within 48 hours of treatment (FIG. 9B). The relative ability of B7-DC XAb, CpG-ODN, and Gardiquimod to inhibit IL-5 production was studied in an experiment in which decreasing amounts of the DC activators were added along with an Ag to spleen cultures of Ag/alum-presensitized spleen cells. On a molar basis, B7-DC XAb (0.06 μg/ml) was 6×10⁴ times more potent than CpG-ODN (6.6 μg/ml) and 10⁵ times more potent than Gardiquimod (2.2 μg/ml) at completely inhibiting IL-5 levels in the cultures as measured by ELISA.

Several controls were included in these experiments that demonstrated that the observed responses were Ag-specific recall responses. When naive spleen cells were pulsed with Ag and incubated with B7-DC XAb, neither IL-4 nor IFN-γ were detected in the 48-hour supernatants. Spleen cells activated with B7-DC XAb but not pulsed with Ag also failed to induce the cytokines, demonstrating that the recall response was Ag driven. Finally, the treatment of DC with Ag together with an IgG Ab (clone TY-25) that binds B7-DC did not induce polarity-modulating activity by the APC. Treatment of spleen cultures with an anti-class II IgM Ab (25-9-3) that binds to the spleen cells together with Ag did not modulate the profile of the cytokines produced. Together, the absence of immune modulatory activity exhibited by three control Abs (sHIgM39, TY25, and 25-9-3) highlight the unique activity of the B7-DC XAb IgM Ab used in these studies.

### Example 9

### Modulation of Recall Response Polarity of Bone Marrow-Derived DC

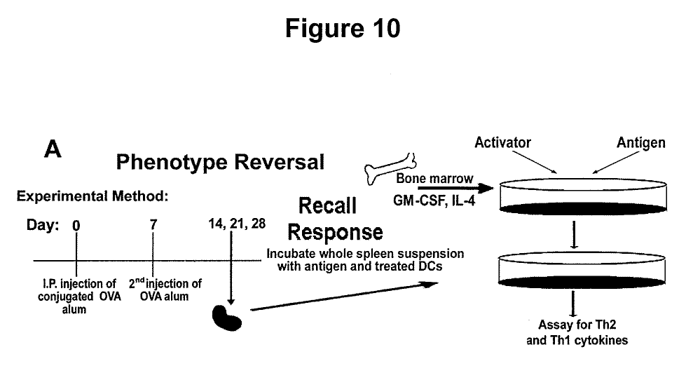

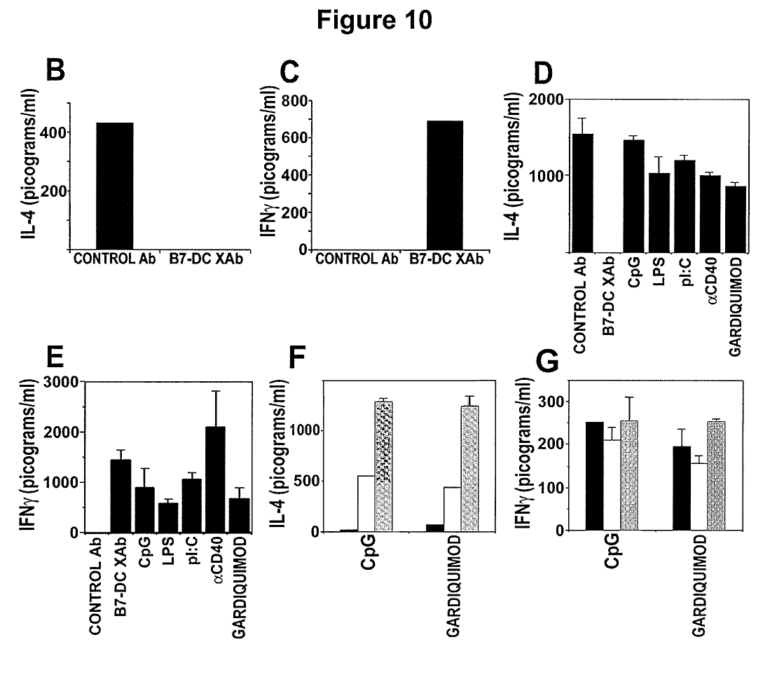

When, instead of adding Ab and Ag directly to a spleen cell culture of Ag-sensitized cells, DC treated in a separate culture for 24 hours with Ag together with B7-DC XAb were added to the spleen cell culture (FIG. 10A), the polarity of the ensuing recall response by the sensitized spleen cells was also modulated from Th2 to Th1 (FIGS. 10B and 10C). Cultures receiving sham-treated DC produced IL-4 and no detectable IFN-γ, while cells receiving DC activated with B7-DC XAb produced IFN-γ and very low levels of IL-4. In contrast, when DC treated with 10 μg/ml CpG-ODN, pI:C, LPS, Gardiquimod, or anti-CD40 Ab were added to the sensitized spleen cell cultures, IL-4 was produced concomitantly with IFN-γ (FIGS. 10D and 10E). Inhibition of IL-4 production was dependent on the concentration of the TLR agonist used to stimulate the DC cultures, approaching the levels achieved with 10 μg/ml B7-DC XAb/ml only when 20 μg/ml TLR agonist was used (FIGS. 10F and 10G).

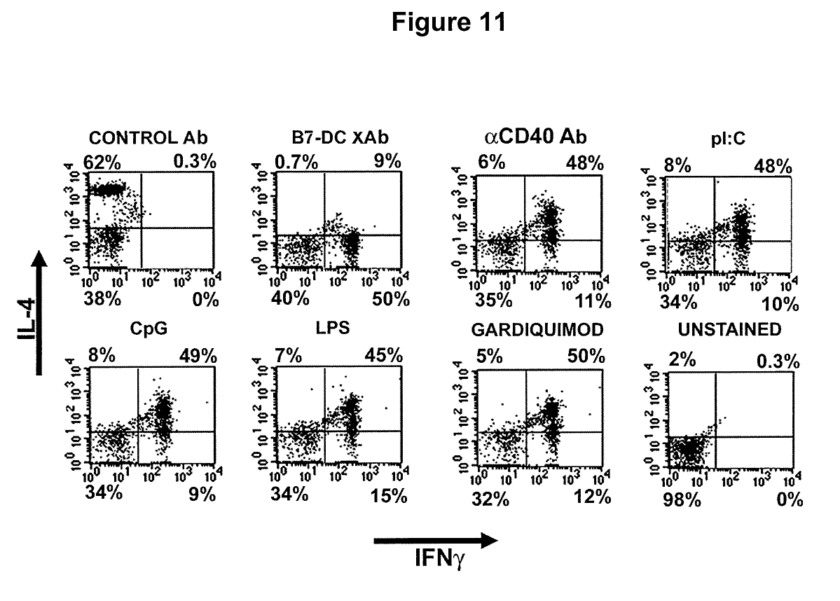

The finding that spleen cell cultures stimulated with TLR and CD40 agonists produced both IL-4 and IFN-γ raised the question of whether the cells were producing both cytokines or whether some responding cells were more sensitive to the modulation of polarity than others. To investigate this question, intracellular levels of cytokines were visualized by flow cytometry following permeabilization of the spleen cell cultures and staining with fluorochrome-labeled Abs specific for IL-4 and IFN-γ. CD4⁺ T cells from cultures modulated with sham control Ab displayed the highest level of staining with IL-4-specific Ab (FIG. 11). After treatment with B7-DC XAb, the levels of IL-4 dropped and the cells expressing IFN-γ predominated In contrast, CD4⁻ T cells from cultures treated with TLR or CD40 agonists displayed an intermediate phenotype with a substantial number of cells staining positive for both IL-4 and IFN-γ following treatment with each of these immune modulators. Few cells were present in the cultures activated with TLR agonists or anti-CD40 that displayed the exclusive IL-4- or IFN-γ-producing phenotypes characteristic of cultures treated with sham modulating Ab or B7-DC XAb.

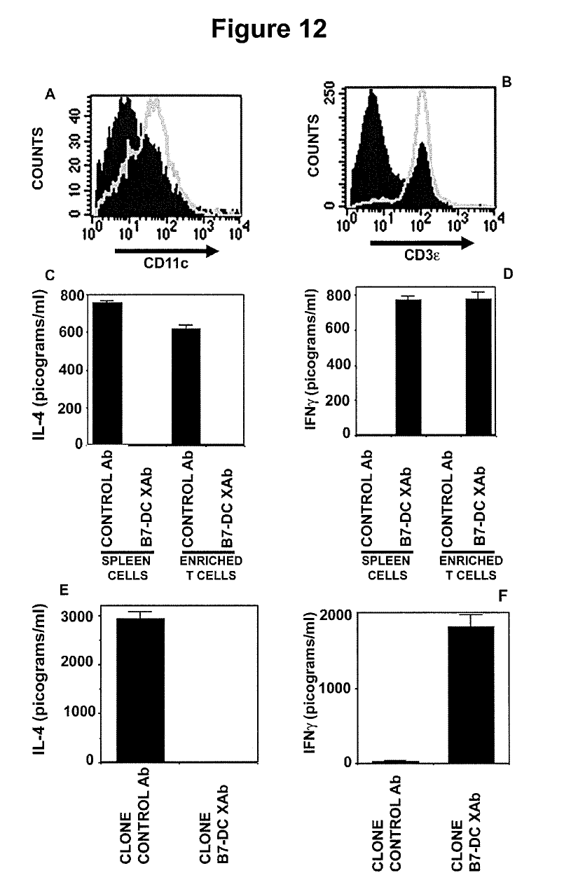

To address whether activated DC were interacting directly with T cells to achieve polarity modulation, two experiments were performed. Bone marrow-derived, Ag-pulsed DC were activated with control Ab or B7-DC XAb and enriched for CD11c⁺ cells (FIG. 12A) and found to be devoid of CD3⁺ cells. These enriched DC were cocultured with purified or cloned Ag-sensitized T cells isolated from OVA/alum-presensitized mice. Purified T cells, 98% pure as judged by flow cytometry (FIG. 12B), responded by producing the Th2 cytokine IL-4 when stimulated with Ag-pulsed DC activated with control Ab (FIG. 12C) and by producing IFN-γ when stimulated with B7-DC XAb Ag-pulsed DC (FIG. 12D). In a second experiment, a long-term established Th2-polarized CD4⁺ T cell clone (McKean et al., supra) specific for sperm whale myoglobulin responded the same way by secreting IL-4 in response to Ag-pulsed, CD11c⁺-enriched DC treated with an isotype control Ab (FIG. 12E) and by switching to the production of IFN-γ when coincubated with DC pulsed with Ag and stimulated with B7-DC XAb (FIG. 12F). Together, these two studies demonstrate that interactions between activated DC and T cells are sufficient to modulate the immune response from Th2 polarity to Th1.

### Example 10

### Requirement for Signal 1 from the Modulating DC During Polarity Modulation of the Splenic Recall Response

The requirement for Ag shown in FIG. 9 implies that responding splenic T cells that undergo the polarity shift must recognize Ag presented by DC in the context of MHC Ag-presenting molecules in the course of the recall response. To evaluate this assumption directly and distinguish between the requirement for Ag to drive the recall response and the possible requirement for Ag to induce a shift in polarity, allogeneic MHC-mismatched DC were pulsed with Ag, washed extensively, and tested for their ability to modulate the polarity of a recall response following coculture with Th2-polarized primed spleen cells. The inability of MHC-mismatched DC to activate the Ag-specific recall response indicates that the recognition of an Ag in the context of MHC molecules on the Ag pulsed DC is required to mobilize the response.

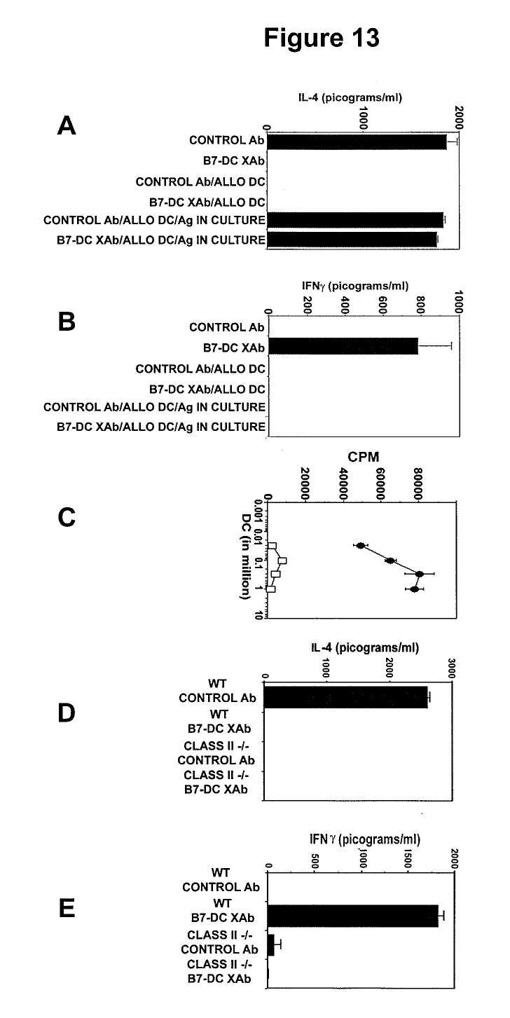

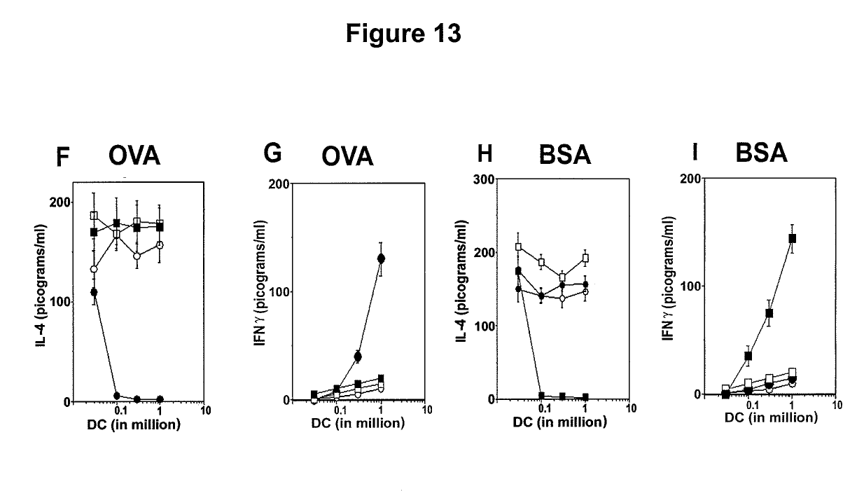

In an independent set of cultures, the spleen cells were also given Ag, providing an independent source of Ag from the Ag presented by the immune-modulating DC. Therefore, the effects of the activated immune modulatory DC on the recall responses were visualized in the context of strong ongoing endogenous signal 1 by DC providing Th2 stimulatory signals. As shown in FIGS. 13A and 13B, the addition of syngeneic Ag-pulsed DC treated with isotype control Ab to sensitized spleen cultures that had either been pulsed or not pulsed with Ag resulted in a strongly Th2-polarized response. When Ag-pulsed DC activated with B7-DC XAb were instead added to comparable spleen cell cultures, even in the presence of endogenous Th2 polarizing signals, a Th1-associated IFN-γ response ensued. In these experiments, naive spleen cells generated neither a Th2 or Th1 response within the 48-hour observation period when treated with B7-DC XAb-activated, Ag-pulsed DC. The ability of the allogeneic DC to be recognized by some elements of the spleen cell culture is indicated by the MLR response visualized by enhanced DNA synthesis in the culture (FIG. 13C).

In an independent set of experiments, B7-DC XAb-activated DC derived from class II deficient mice were used to probe the MHC dependence of the observed immune modulatory effects. In these experiments, DC were derived from class II-knockout bone marrow, pulsed with Ag, and treated with B7-DC XAb or control Ab overnight. The DC were then added to spleen cell cultures from OVA/alum-presensitized mice to determine whether the ability of DC to modulate the polarity of the recall response was class II dependent. As shown in FIGS. 13D and 13E, class II-deficient DC were unable to induce a recall response, confirming the importance of MHC/TCR interactions in this process. It can be concluded from these experiments that sensitized Th2-polarized T cells must receive signal 1 to be reprogrammed by B7-DC XAb-activated DC to respond with Th1 polarity.

The Ag specificity of polarity modulation was observed in animals immunized either with OVA or BSA. Spleen cell cultures were rechallenged with the appropriate presensitizing Ag, OVA, or BSA along with increasing numbers of isotype control-treated or B7-DC XAb-treated DC pulsed with one Ag or the other. As shown in FIGS. 13F-13I, the polarity shift induced by B7-DC XAb-activated DC was Ag specific in that only DC pulsed with the sensitizing Ag could mediate the shift. Down-regulation of IL-4 and up-regulation of IFN-γ occurred at approximately the same point in the titration curves for both Ags in cultures receiving varying numbers of Ab-stimulated DC.

B7-DC XAb polarization of the T cell response is long lived in that spleen cells isolated more than 8 weeks after Ab treatment responded with a Th1 phenotype when rechallenged with Ag. Immunization of mice with the unrelated Ag BSA in the context of the Th2-polarizing adjuvant alum resulted in cells that responded to a rechallenge with BSA by producing IL-4 and no IFN-γ. This demonstrated that B7-DC XAb modulates only the response to the antigens present at the time of treatment, and does not alter the polarity of subsequent responses that are elicited in the absence of the immune modulator.

### Example 11

### Contribution of Adhesion Molecules to DC-Mediated Modulation of Recall Response Polarity

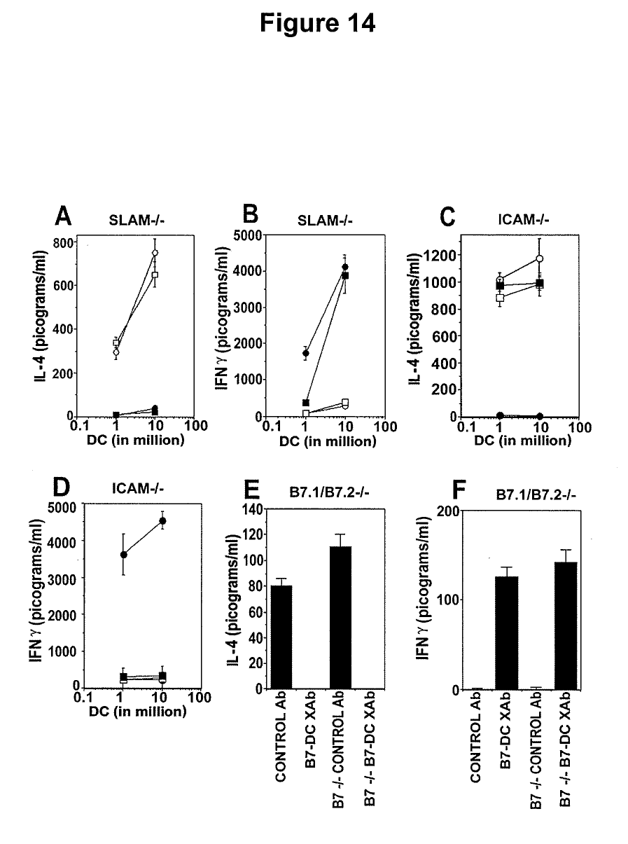

The finding that Ag recognition of activated DC is required to support the reprogramming of the cytokine response by sensitized T cells implies that cell-to-cell contact may be an important aspect of communication between these cells. Treatment of DC with B7-DC XAb resulted in up-regulation of the adhesion molecules ICAM-1, SLAM, and LY-9 (Tseng et al. (2001) J. Exp. Med. 193:839-846). To evaluate the importance of molecules known to function in communication between APC and T cells, DC genetically deficient in key regulatory molecules were assessed for their ability to modulate the polarity of recall responses. The importance of SLAM expression in supporting the modulation of the polarity of the recall response is indicated by relative inefficiency of SLAM-deficient DC using the standard numbers of APC and spleen cells to promote a Th1 response as compared with wild-type DC. Initial studies indicated that DC deficient in SLAM expression down-regulated IL-4 expression but did not induce IFN-γ. This same effect (IL-4 inhibition in the absence of induced IFN-γ) can be achieved by reducing the number of Ag-pulsed wild-type DC activated with B7-DC XAb added to the spleen cultures by 10-fold (FIGS. 13F-13I). To evaluate whether this deficiency to induce IFN-γ was quantitative or qualitative in nature, the number of SLAM-deficient DC added to the spleen cultures was varied. Under these conditions, IFN-γ was not induced when the standard number of DC were added (10⁶) but was induced when the number of APC was increased 10-fold (FIGS. 14A and 14B). Therefore, although the expression of SLAM is not required for polarity reprogramming, this costimulatory adhesion molecule facilitates the process. In contrast, the DC deficient for the SLAM homologue LY9 were just as effective as wild-type DC in modulating the polarity of the recall response. Antigen-pulsed, ICAM-1-deficient DC failed to down-regulate IL-4 production or up-regulate IFN-γ when added to sensitized, Th2-polarized spleen cells, irrespective of whether 10⁶ or 10⁷ DC were used (FIGS. 14C and 14D). These data support the view that SLAM expression by DC enhances the efficiency of DC to promote a polarity switch of activated/memory T cells, while ICAM-1 is critical for this function.

### Example 12

### CD80/CD86 Costimulation is Not Required for Modulation of Recall Response Polarity

Consistent with the nature of recall responses, expression of the costimulatory molecules B7.1 (CD80) and B7.2 (CD86) by B7-DC XAb-activated DC was not required to induce a Th1-polarized recall response by sensitized spleen cells (FIGS. 14E and 14F). This is in contrast to the requirement of these important costimulatory molecules for the generation of sensitized T cells in first place.

### Example 13

### Requirement for a Signal 3 to Modulate Recall Response Polarity

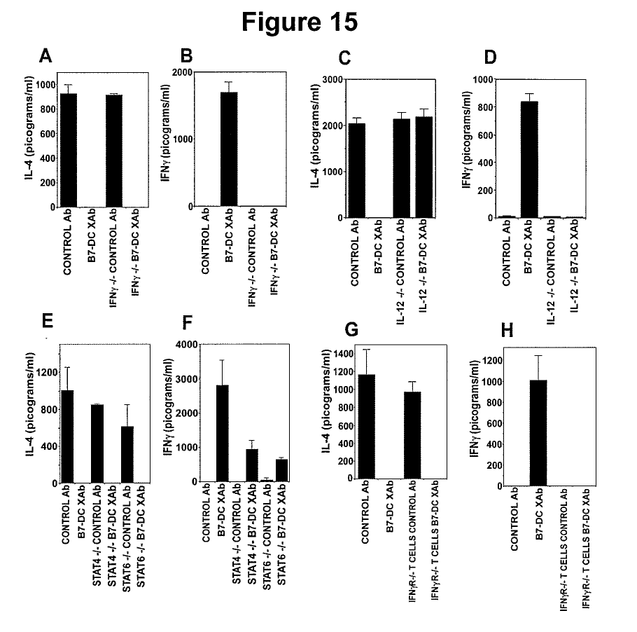

Experiments were conducted to examine other possible signals emanating from activated DC that could determine the ability of these cells to efficiently direct a change in the polarity of responding sensitized T cells. The inventors have found that bone marrow-derived DC activated with B7-DC XAb secrete IL-6, TNF-α, IFN-γ, and IL-12 (see, e.g., Table 1). To investigate whether these cytokines could influence the polarity of induced recall responses, DC deficient in their ability to express these cytokines were evaluated for their ability to direct a Th1-polarized recall response in vitro. Although IL-6- and TNF-α-deficient DC functioned just as efficiently as wild-type DC, significant changes were observed when IFN-γ- and IL-12-deficient DC were used (FIGS. 15A-15D). Although B7-DC XAb-activated, IFN-γ-knockout DC were able to down-regulate IL-4 production, they were not able to induce IFN-γ production by sensitized wild-type spleen cells. B7-DC XAb-activated, IL-12-knockout DC neither down-regulated IL-4 nor up-regulated IFN-γ production, even when 10-fold more DC were added to the cultures. This indicates that the cytokines produced by activated DC are important factors in programming the polarity of the recall response.

To assess the possible importance of Th1- or Th2-polarizing cytokines on DC function, DC deficient in the expression of the STAT4 and STAT 6 transcription factors were activated with B7-DC XAb and tested for their ability to modulate the polarity of recall responses by wild-type sensitized spleen cells. As shown in FIGS. 15E and 15F, DC derived from STAT4- and STAT6-knockout mice were able to modulate the recall response of OVA-sensitized spleen cells, inhibiting expression of IL-4 and inducing expression of IFN-γ. Because the IL-12 receptor signals through a STAT4-dependent pathway, the finding that DC derived from STAT4 deficient mice can induce a polarity switch by sensitized spleen cell cultures indicates that IL-12 is not likely acting on the B7-DC XAb-activated DC but rather functions on responding T cells. Similarly, the requirement for IFN-γ production by the activated DC to induce IFN-γ secretion by spleen cells during the recall response implies an important role for IFN-γ receptors on responding Ag-specific T cells. This hypothesis is supported by the inability of IFN-γ receptor-deficient Ag-sensitized spleen cells to produce IFN-γ in a recall response induced by wild-type B7-DC XAb-activated DC (FIGS. 15G and 15H).

In summary, the data presented above support a method for modulating immune responses by manipulating DC in an Ag-independent fashion, allowing the responses to a variety of antigens to be modified using a common treatment scheme.

### Example 14

### Materials and Methods for Examples 15-22

Mice and reagents: C57BL/6J mice, BALB/CJ mice, Class II restricted ovalbumin peptide specific TCR transgenic mice, DO11.10, Class I restricted ovalbumin peptide specific TCR transgenic mice, OT-II, and IL-6 knock out mice (B6.129S2-IL6tm1Kopf/J) were purchased from The Jackson Laboratory. BALB-neuT mice (H-2d) were bred in Mayo Clinic facilities. Heterozygous 6- to 15-week-old females expressing rat HER-2/neu were tested by PCR before being used in experiments. The P815 mastocytoma cell line (H-2d) was purchased from the American Type Culture Collection (ATCC; Manassas, Va.). The mouse breast cancer cell line transfected with rat HER-2/neu, A2L2, untransfected parental cell line, 66.3, and a cloned cell line from established carcinoma that spontaneously arose in a Balb-neuT mouse, TUBO, were a kind gift from Dr. Celis, Moffitt Cancer Center, Tampa, Fla. Peptide p66 (TYVPANASL; SEQ ID NO:13) generated from RNEU antigen was synthesized in the Mayo Protein Core Facility. The immunostimulatory CpG oligonucleotide, ODN-1826 (5′-TCCATGACGTTCCTGACGTT-3; SEQ ID NO:12), was prepared by the Mayo Clinic Molecular Core Facility. The hybridoma clone PC61 secreting antibody against CD25 was purchased from ATCC. The antibody was purified from supernatants using a protein A sepharose column. The MTAbs, sHIgM39 (isotype matched control antibody or MTAb39) and sHIgM12 (also referred to as MTAb12 or B7-DC XAb), were purified from human serum as described in Example 1. Anti-mouse CD4-PE (RM4-5) antibody and anti-mouse IFNγ-PE (XMG1.2) antibody were purchased from BD Biosciences (San Jose Calif.). Anti-mouse FoxP3-APC (FJK-16s), anti-DO11.10 TCR-FITC (KJ1-26), anti-mouse TNFα-PE (MP6-XT22), anti-mouse IL-10-PE (JES5-16E3) and anti-mouse IL-17A-PE (eBio17B7) antibody were purchased from eBioscience (San Diego, Calif.). Carboxyfluorescein diacetate succinimidyl ester (CFSE) was purchased from Molecular Probes (Carlsbad, Calif.) and stored as 10 mM stock at −20° C.

Immunization protocol: Immunization and analysis of the ensuing immune response was carried out as follows. Briefly, mice (BALB/c or BALB-neuT) received five daily s.c. injections of 100 μg CpG (days −2, −1, 0, 1, and 2). On day 0, mice were immunized (s.c.) with 100 μg p66 peptide emulsified in IFA (200 μL) at a proximal site of the CpG injections. For the in vivo Treg cell depletion experiments, anti-CD25 mAb (PC61) (0.5 mg per mouse) was injected i.p. on days −3, −2, and −1 before the peptide injection. Some groups of mice were injected with 10 μg of MTAb, sHIgM39 control IgM antibody or MTAb 12, B7-DC XAb on days −1, 0, and +1 as intra peritoneal injections. Mice were challenged with 2×10⁶ TUBO tumor cells subcutaneously.

Elispot assay: Immune responses were measured using enzyme-linked immunosorbent spot (ELISPOT) assays to detect the frequency of CD8 T cells secreting IFN-γ (Mabtech; Mariemont, Ohio). Serial dilutions of purified CD8 T cells (Miltenyi Biotec; Auburn, Calif.) were tested against a constant number (3×10⁵) of stimulator cells. The number of spots was counted using an AID ELISPOT Reader System (Cell Technology; Columbia, Md.), and the data are presented as ratio of number of spots to CD8 T cells.

Isolation of Tregs and non-Tregs: Splenocytes were harvested from different groups of mice and Tregs were isolated by positive selection using the Mouse Treg Isolation kit from Miltenyi Biotec as per the manufacturer's protocol. Briefly, cells were incubated with anti-CD25-PE antibody for 10 minutes. After washing with MACS buffer, anti-PE magnetic beads were added and cells were incubated for 15 minutes. Cells were washed and passed through the magnetic column. The flow-through cells were referred to as Non-Tregs (CD25−), while, the cells obtained upon flushing from the column were referred to as Tregs (CD25+).

Generation of bone marrow dendritic cells: DC bone marrow of wild type mice or IL-6^{-/-} mice was isolated using an established protocol. Briefly, bone marrow was retrieved from the long bones of the hind legs. Lysis of red blood cells was achieved by treatment with ammonium chloride/potassium bicarbonate/EDTA at 37° C. Cells were plated at the density of 1×10⁶/ml in six-well plates (BD Biosciences; San Jose, Calif.) in RPMI 10 containing 10 μg/ml of murine GM-CSF and 1 ng/ml of murine IL-4 (PeproTech; Rocky Hill, N.J.). At day 2 of culture, floating cells were gently removed and were replaced with RPMI 10 containing the same concentration of GM-CSF and IL-4 for another 5 days. Antigen pulse and MTAb 39 or MTAb12 IgM antibody treatment was carried out on day 6, followed by overnight incubation. Cells were washed at day 7 before being used to stimulate enriched Tregs or Non-Tregs. In experiments involving matured dendritic cells, 50 μM CpG was added into day 6 cultures along with the antigen for overnight incubation. At day 7, cells were washed and used as stimulators.

In vitro and in vivo activation of Tregs and non-Tregs: Bone marrow derived wild type or IL-6^{-/-} immature dendritic cells that were antigen-pulsed and MTAb39 or MTAb12 IgM antibody treated, or CpG matured dendritic cells were used to stimulate naïve D011.10 Tregs or OT-II Tregs at a 1:1 ratio for 48 hours. Cells were harvested and washed twice in PBS before being used for multiple analyses. In vivo activation of Tregs was achieved by adoptive transfer of D011.10 or OT-II derived Tregs into haplotype matched mice that also received 3×10⁶ antigen-pulsed, MTAb39 or MTAb12 IgM antibody treated bone marrow derived dendritic cells. In experiments involving modulation of endogenous dendritic cells to activate Treg cells, D011.10 or CFSE labeled OT-II Treg cells were transferred into mice along with administration of MTAb39 or MTAb12 antibody and 100 μg of ovalbumin as antigen. After 48 hours, spleen cells were harvested and analyzed by flow cytometry. A clonotypic TCR antibody, KJ1-26, was used to mark D011.10 Tregs, while CFSE was used to mark OT-II Tregs.

Cell proliferation assay: To monitor the proliferative ability of Tregs, CFSE labeled DO11.10 Tregs or OT-II Tregs were adoptively transferred along with antigen-pulsed MTAb39 or MTAb12 treated DC. After 48 hours, splenocytes were analyzed for CFSE dilution by flow cytometry. In experiments involving measurement of [³H] incorporation, Tregs or non Tregs from D011.10 mice were stimulated with serially diluted antigen-pulsed MTAb39 or MTAb12 treated DC for 72 hours. Alternatively, in suppression experiments, Tregs that were stimulated with antigen-pulsed MTAb39 treated DC or MTAb12 treated DC for 48 hours were titrated with a constant number of D011.10 non Treg cells and antigen-pulsed DC. Cultures were pulsed with [³H] thymidine overnight before harvesting and counting.

Elisa for TGFβ1: Culture supernatant collected from wells containing DC and Tregs for 48 hours was measured by ELISA for TGFβ1 using a kit from R&D Systems (Minneapolis, Minn.) as per the manufacturer's protocol. Briefly, activation of the samples was carried out by incubating 100 μl of supernatant with 20 μl of 1N HCL for 10 minutes at room temperature. The acid was neutralized by addition of 20 μl of 1.2N NaOH/0.5M HEPES. Samples were subsequently assayed for the amount of TGFβ1 using standard sandwich ELISA.

Quantitative RT-PCR for FoxP3: Non-Tregs and Tregs were isolated as described above. Isolated cells were stimulated with antigen-pulsed dendritic cells treated with MTAb39 or MTAb12 antibody. After 48 hours, RNA from cells of all groups was isolated using Trizol reagent. 300 ng of template RNA were mixed with mouse FoxP3 primers (Forward primer 5′-CTACTTCAAGTACCACAATATGCGAC-3′; SEQ ID NO:14; Reverse primer 5′-CGTTGGCTCCTCTTCTTGCGAAACTC-3′; SEQ ID NO:15) or actin primers (Forward primer 5′-CGTCTGGACTTGGCTGGCCGGGACCT-3′; SEQ ID NO:16; Reverse primer 5′-AGTGGCCATCTCCTGCTCGAAGTCTA-3′; SEQ ID NO:17). Quantitative PCR was performed using Applied Biosystems' 7900HT real-time PCR instrument. Power SYBR Green from Applied Biosystems (Foster City, Calif.) was used for detection and quantification was carried out using SYBR green kit.

CFSE labeling of Tregs: Tregs isolated from appropriate mice were washed twice in PBS to remove serum proteins. Cells were resuspended in 5 ml PBS, CFSE was added to a final concentration of 5 μM, and cells were incubated at 37° C. for 15 minutes. After washing twice in 10% FBS, cells were plated and analyzed after 48 hours by flow cytometry.

Flow cytometry: Tregs or Non-Tregs that were recovered after stimulation were washed with FACS buffer (0.5% BSA and 0.1% sodium azide in PBS) and centrifuged into a 96-well plate Nunc (Rochester, N.Y.) for staining the markers on the surface of the cell. Abs were added to the wells for a 30 minute incubation on ice. After three washes, cells were subjected to intracellular staining using BD Cytofix/Cytoperm kit for BD Biosciences (San Jose, Calif.). Briefly, cells were incubated with Cytofix solution for 20 minutes on ice. After washing the cells with Cytoperm buffer, antibody against indicated intracellular proteins were diluted in Cytoperm buffer and cells were incubated with the diluted antibody for 30-min on ice. Cells were subsequently washed with FACS buffer and run on a FACSCalibur flow cytometer BD Biosciences (San Jose, Calif.). Data were analyzed using CellQuest software BD Biosciences.

Multiplex cytokine immunoassay: Culture supernatants obtained from D011.10 Tregs stimulated with MTAb39 or MTAb12 treated, antigen-pulsed DC, or non-Tregs that were stimulated with antigen-pulsed MTAb39 or MTAb12 treated dendritic cells for 48 hours, were assayed for an array of cytokines using a multiplex cytokine assay. Additional controls that were tested included supernatants from dendritic cells alone that were stimulated with MTAb39 or MTAb12. A Bio-Rad mouse cytokine panel (Bio-Rad Laboratories; Hercules, Calif.) was used for this study. The assay was performed as per the manufacturer's protocol along with standards. Briefly, 100 ml of Bio-Plex assay buffer were added to each well of a MultiScreen MABVN 1.2-μm microfiltration plate, followed by addition of 50 μl of multiplex bead preparation. After washing the beads with the addition of 100 μl wash buffer, 50 μl of the samples or standards were added to each well and incubated with shaking for 30 minutes at room temperature. The plate was then washed three times, followed by incubation of each well in 25 μl of premixed detection Abs for 30 minutes with shaking. The plate was further washed and 50 μl of streptavidin solution was added to each well and incubated for 10 minutes at room temperature with shaking. The beads were washed and resuspended in 125 μl of Bio-Plex assay buffer. Cytokine levels in the culture supernatant were quantitated by analyzing 100 μl of each sample on Bio-Plex using Bio-Plex Manager software, version 4.0. Standard curves were generated with a mixture of cytokine standards provided by the supplier and eight serial dilutions ranging from 0 to 32,000 pg/ml.

Toxicology studies: On day one, twenty 8 week old male C57BL/6J mice were weighed, transferred to individual cages and ten mice were injected i.v. with 300 μg B7-DCXab in 100 μl PBS while another 10 mice were injected with 100 μl PBS. Treatment was blinded until the end of the experiment. Mice were observed daily and weighed again on days 7 and 14. On day 14, the mice were shipped from Mayo Clinic Rochester, Minn. to Mayo Clinic Scottsdale, Ariz. The mice arrived in Scottsdale on day 16, at which time they were fasted overnight. On day 17, the mice were visually evaluated and weighed, and blood samples were collected for hematology and chemistry analyses. Hematology analysis included determining the numbers and percentages of white blood cells, lymphocytes, granulocytes, monocytes, and red blood cells, as well as hematocrit, mean corpuscular volume (MCV), hemoglobin, mean corpuscular hemoglobin (MCH), mean corpuscular hemoglobin with concentration (MCMC), red cell distribution width (RDW), mean platelet volume (MPV), and platelet counts. The serum chemical analysis included blood urea nitrogen (BUN), glucose and creatine levels, Alkaline phosphatase (ALP) and alanine aminotransferase (ALT), and T-Pro measurements. Following the blood collection, the mice were euthanized and the heart, brain, spleen, kidney, liver, and lungs were harvested in 10% buffered formalin for histopathology.

### Example 15

### B7-DC XAb Renders Tolerant Tregs Responsive

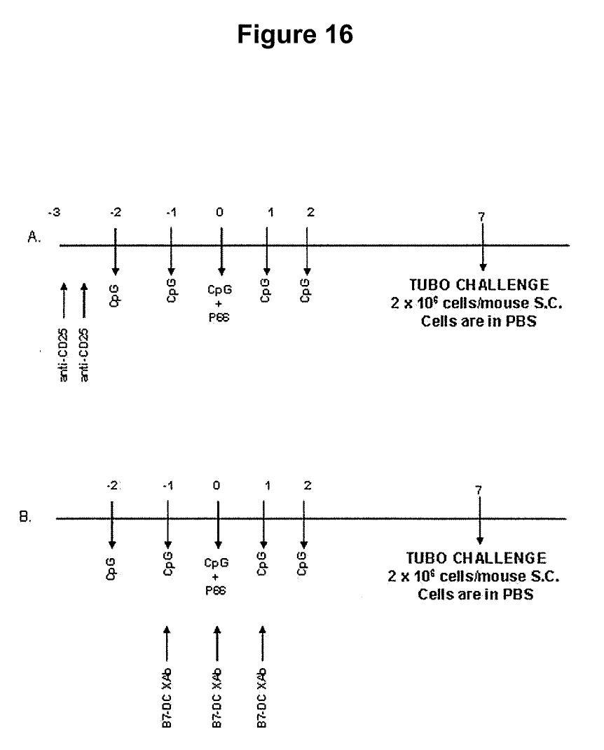

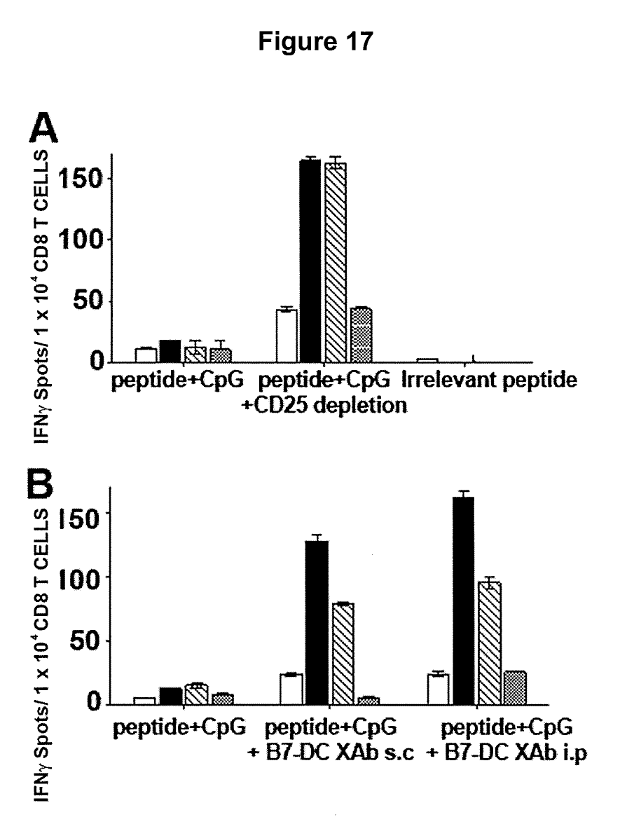

Elimination of Tregs by systemically treating BALB/c-NeuT (rat Her2neu transgenic) animals with CD25-specific antibodies (FIG. 16) rendered tolerant mice responsive to the immunodominant rat Her2/neu peptide p66 (FIG. 17). Treatment with the immune modulatory human IgM antibody B7-DC XAb also promoted responsiveness against the p66 peptide by otherwise tolerant BALB/c-NeuT mice (FIG. 17). The similarities in these response patterns suggested the possibility that treatment with B7-DC XAb modulates the function of Tregs. The possibility that immune modulation using the MTAb B7-DC XAb might provide an antigen-specific method to down regulate Treg function could have profound implications for cancer immunotherapy and the treatment of chronic virus infection.

### Example 16

### Ablation of FoxP3 on CD25+ CD4+ Tregs in the Presence of B7-DC XAb

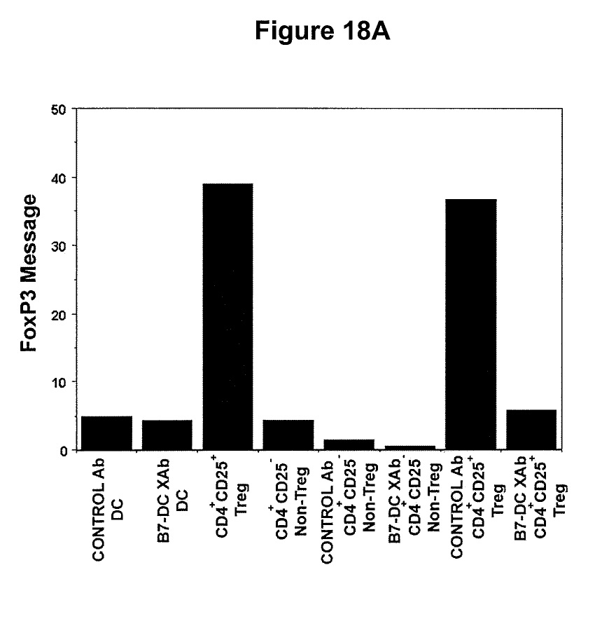

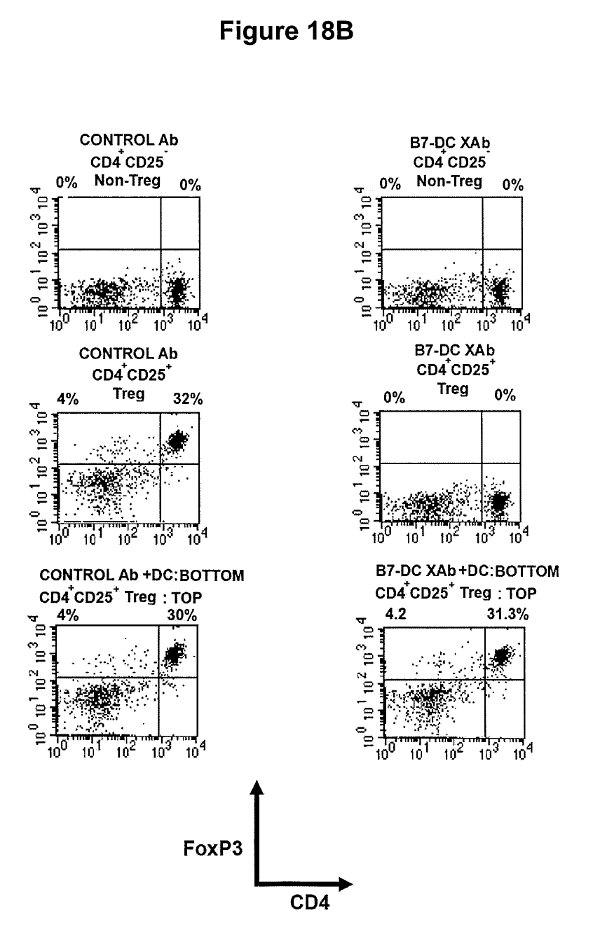

To evaluate the effects of DC activated with B7-DC XAb on Tregs, enriched CD4+CD25+ splenic T cells from DO11.10 mice were co-cultured with OVA-pulsed B7-DC XAb-activated DC for 48 hours and assayed for FoxP3 message. Tregs cultured with antigen-pulsed B7-DC XAb-activated DC markedly down regulated FoxP3 mRNA in comparison to message levels in Tregs incubated with antigen-pulsed DC treated with a control human IgM (FIG. 18A).

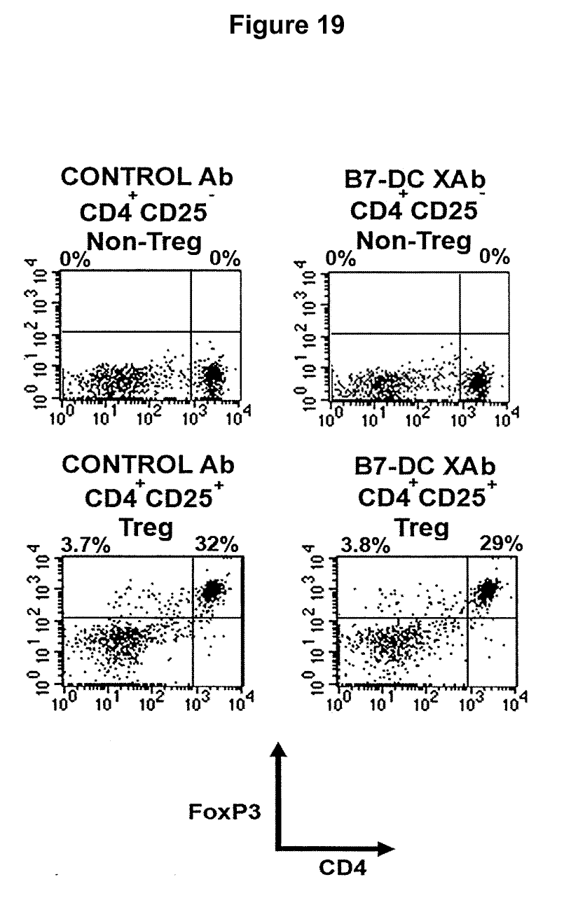

Suppression of FoxP3 expression could also be seen at the protein level. Enriched CD25+CD4+ DO11.10 T cells (Tregs) and CD25-CD4+DO11.10 T cells (non-Tregs) were stimulated with ovalbumin pulsed B7-DC XAb or control antibody treated DC for 48 hours and the cells were analyzed for FoxP3 expression by intracellular flow cytometry. As shown in FIG. 18B, B7-DC XAb-activated antigen-pulsed DC suppressed the expression of FoxP3 protein by CD4+ DO11.10 Tregs. Importantly, B7-DC XAb-activated DC not pulsed with antigen were unable to suppress FoxP3 protein (FIG. 19). Therefore, modulation of the Treg phenotype with B7-DC XAb is antigen-dependent. Furthermore, a requirement for cellular contact between the B7-DC XAb-activated DC and the Tregs was evident in experiments using Boyden chambers.

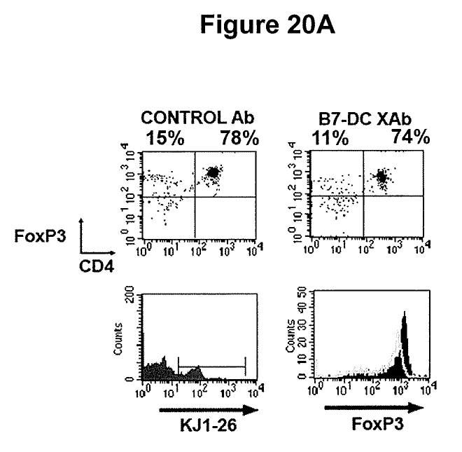

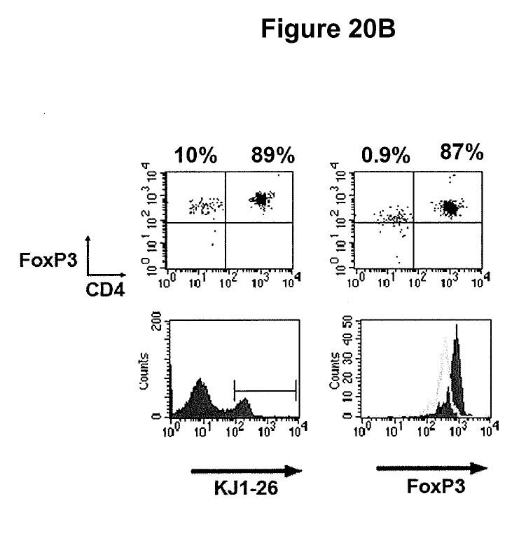

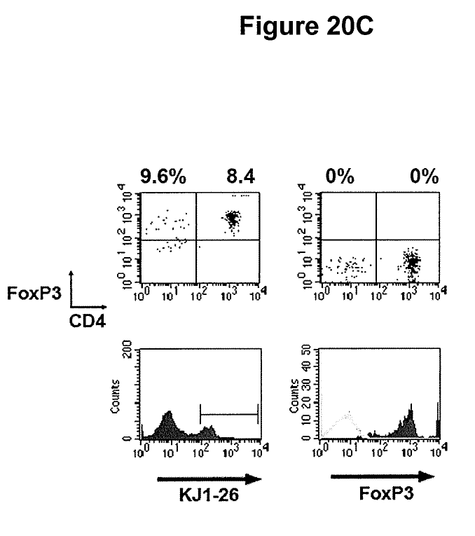

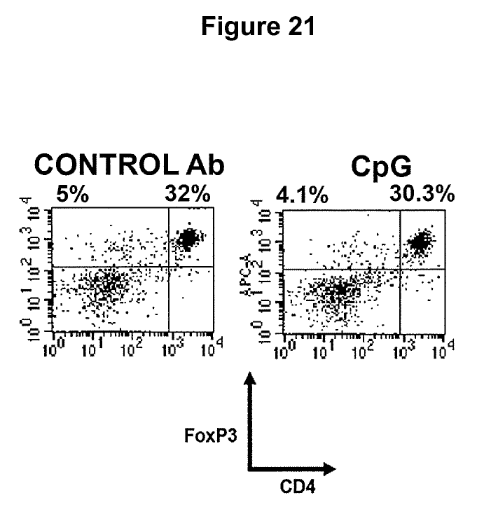

Tregs from DO11.10 mice that were stimulated with B7-DC XAb treated DC and antigen were analyzed for expression of FoxP3 over a 48 hour time course. Modest down regulation was observed by 36 hours, with complete down regulation by 48 hours (FIG. 20). Thus, B7-DC XAb-treated, antigen-pulsed DC were able to induce down regulation of FoxP3 in Tregs within 2 days. In contrast to what was observed with B7-DC XAb-activated DC, DC matured with the TLR-9 agonist CpG did not down regulate FoxP3 expression by CD4+CD25+ D11.10 T cells in similar cultures (FIG. 21).

### Example 17

### Down Regulation of FoxP3 by B7-DC XAb In Vivo is Antigen Specific

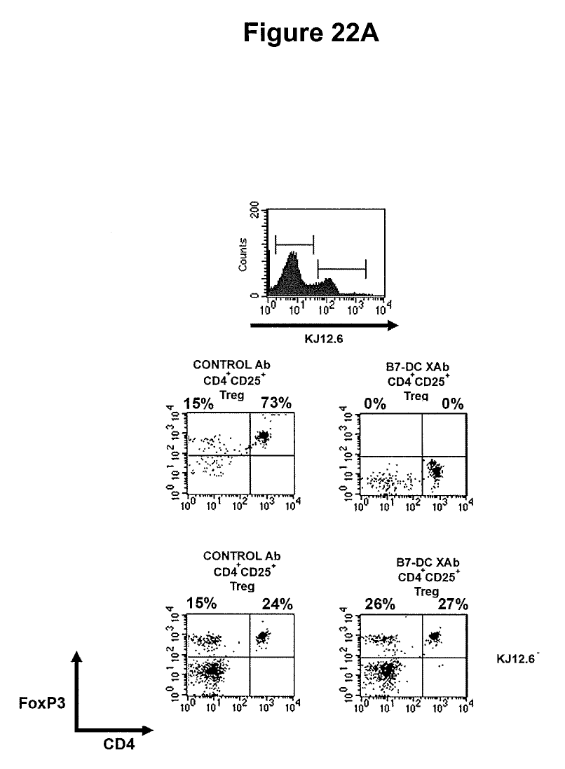

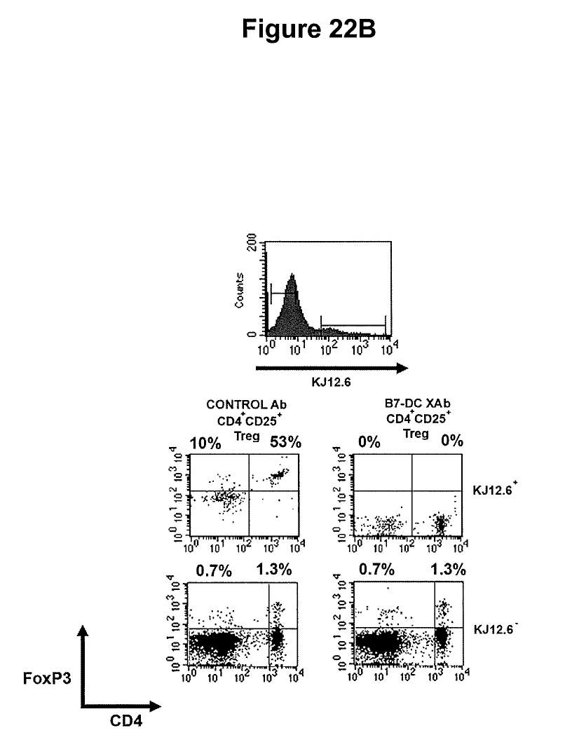

The effects of cross linking B7-DC resulting in alterations of Treg in vitro in the presence of bone marrow derived DC is antigen-dependent. To address whether down modulation of the Treg phenotype is antigen-specific, experiments were conducted to assess the ability of B7-DC XAb to modulate the phenotype of antigen-specific DO11.10 Tregs mixed in culture with syngenic BALB/cJ Tregs. OVA pulsed DC treated with isotype control antibody or B7-DC XAb were added to the cultures. After 48 hours, splenocytes were harvested from the recipients and analyzed for expression of FoxP3. BALB/c Tregs and the ovalbumin-specific D011.10 Tregs were distinguished by the presence or absence of the KJ1-26 clonotypic marker expressed by DO11.10 T cells. As shown in FIG. 22A, recovered DO11.10 T cells treated with antigen-pulsed DC incubated with isotype control antibody retained FoxP3 expression, while DO11.10 T cells recovered from culture receiving DC activated with B7-DC XAb did not. Importantly, expression levels of FoxP3 were normal in the BALB/c CD4+CD25+Tregs in both groups, demonstrating that down regulation of FoxP3 by B7-DC XAb-activated DC was antigen specific.

Studies were then conducted to determine whether endogenous DC can mediate the downregulation of Tregs in vivo when activated with B7-DC XAb. BALB/cJ mice received adoptively transferred DO11.10 Tregs, ovalbumin, and isotype control antibody or B7-DC XAb. Spleen cells were recovered 48 hours later and stained for expression of the DO11.10 clonotypic TCR, CD4, and FoxP3. As shown in FIG. 22C, administration of antigen with B7-DC XAb, but not with control antibody resulted in the down regulation of FoxP3 expressed by DO11.10 T cells in vivo, while endogenous Tregs retained FoxP3 expression (FIG. 22B).

### Example 18

### Down Regulation of FoxP3 Following Treatment with B7-DC XAb Results in Loss of Suppressive Activity by DO11.10 CD4+CD25+ T Cells

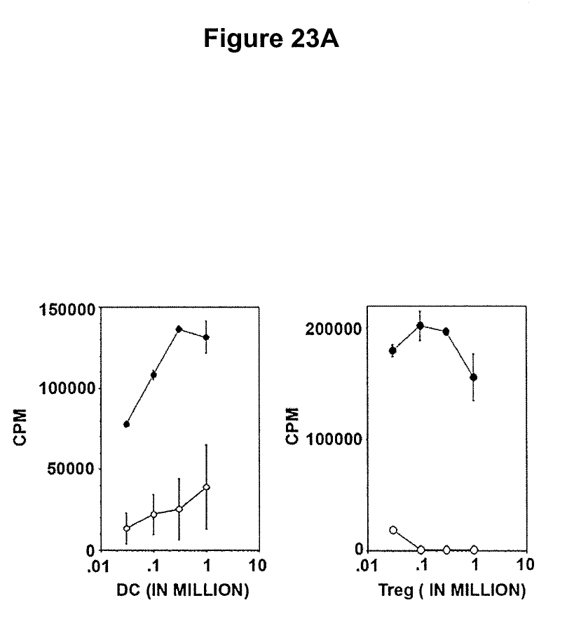

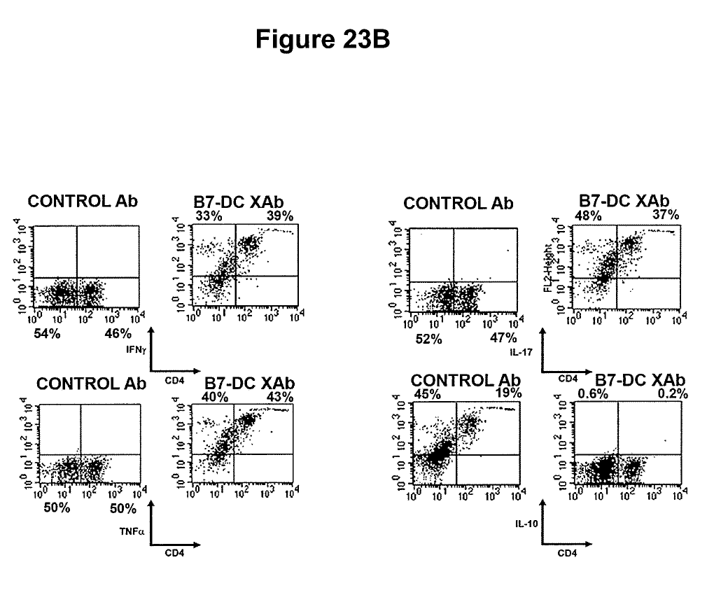

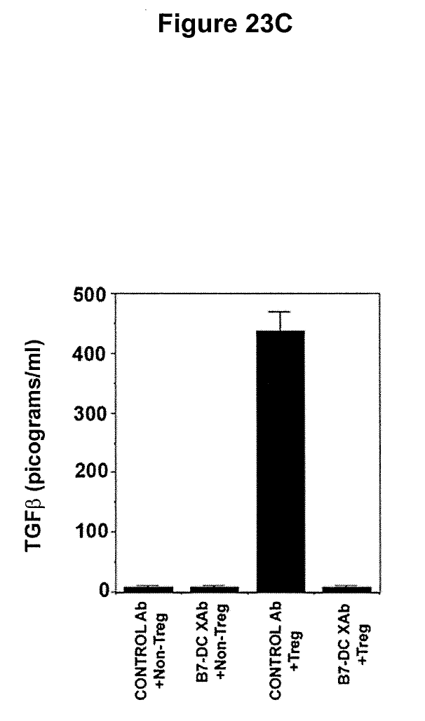

To assess the functional consequences of down regulation of Foxp3 in Tregs by antigen-pulsed DC activated with B7-DC XAb, Tregs isolated from DO11.1 mice and stimulated in vitro with antigen-pulsed DC treated with antibody were evaluated for their ability to suppress antigen-specific responses by effector T cells. In these studies, Tregs activated with antigen-pulsed DC treated with isotype control antibody proliferated minimally. In contrast, CD4+CD25− T effector cells stimulated with antigen-pulsed DC activated with B7-DC XAb proliferated robustly (FIG. 23A). Tregs (stimulated with control antibody-treated DC and antigen) suppressed proliferation of antigen-stimulated CD4+CD25− DO11.10 T cells, while Tregs modulated with antigen-pulsed DC activated with B7-DC XAb did not.

Tregs secrete IL-10 and TGFβ, cytokines capable of suppressing T cell responses. The array of cytokines produced by Tregs stimulated with B7-DC XAb treated DC or control antibody treated DC was determined using a bead-based cytokine assay. IFNγ, TNFα and IL17 were found at increased levels in cultures containing B7-DC XAb-activated, antigen-pulsed DC and DO11.10 Tregs, while the immunosuppressive cytokine IL-10 was down regulated (Table 2). To determine whether these cytokines were emanating from the T cells in the cultures, DO11.10 Tregs were recovered and analyzed by intracellular flow cytometry. In concordance with the multiplex analysis, IFNγ, TNFα, and IL-17 were up regulated in the CD4 T cells, while, IL-10 and FoxP3 were down regulated (FIG. 23B). The flow cytometry profiles in FIG. 23 suggest that the recovered T cells were likely simultaneously producing all three cytokines. This pattern was confirmed in double labeling experiments demonstrating that cells secreting IFNγ also were secreting TNFα, and IL-17 (FIG. 20). TGFβ in the culture supernatants was down regulated as well, as visualized by sandwich ELISA (FIG. 23C). Taken together, these studies indicate Tregs lose the expression of FoxP3, TGFβ, and IL-10, the ability to suppress T effector cell responses, but gain the ability to produce the proinflammatory cytokines IL-17, IFNγ and TNFα when modulated by B7-DC XAb-activated DC.

TABLE 2
Multiplex cytokine analysis on Tregs stimulated with B7-DC XAb
treated DC

| | Treg + MTAb39 DC | Treg + MTAb12 DC |
|---|---|---|
| Cytokines | (ng/ml) | (ng/ml) |
| IFNγ | 1.39 ± 0.12 | 28.5 ± 0.7 |
| TNFα | 0.3 ± 0.00 | 29 ± 0.1 |
| IL-17 | 6.07 ± 0.12 | 29.6 ± 0.35 |
| IL-10 | 30.2 ± 0.2 | 0.7 ± 0.09 |

### Example 19

### Modulation of Treg Phenotype by B7-DC XAb can Occur in Absence of Treg Proliferation

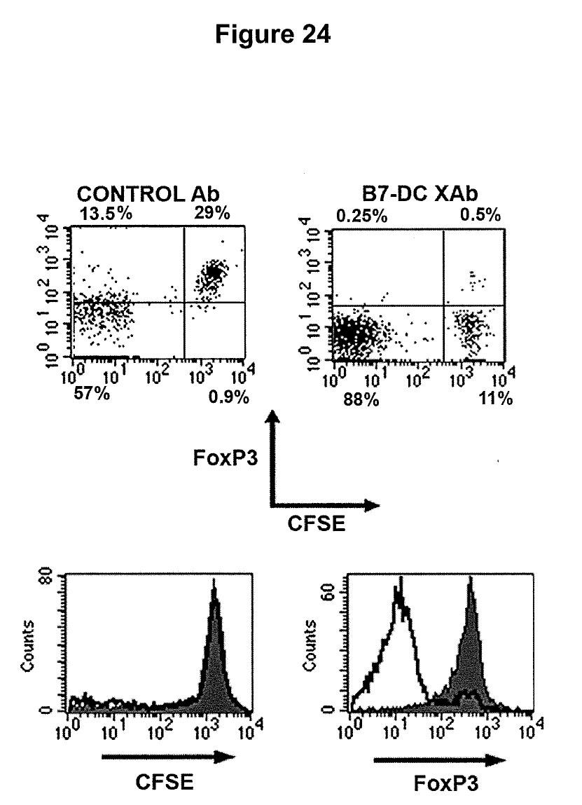

Alterations in phenotype and functional characteristics of Tregs have been demonstrated both in vivo and in vitro following activation by antigen-pulsed, B7-DC-treated DC. Not withstanding efforts to use enriched cell populations to visualize the immune modulatory effects of B7-DC XAb, the possibility was considered that the apparent modulation of the Treg phenotype was actually the consequence of the expansion of non-Tregs effectors remaining in the populations of enriched Tregs. Accordingly, Tregs were labeled with CFSE and stimulated with antigen-pulsed, control antibody-treated or B7-DC XAb-activated DC for 48 hours prior to characterization. As shown in FIG. 24A, Tregs remained CFSE high and FoxP3 high when stimulated with isotype control antibody treated DC in presence or absence of antigen. Upon treatment with antigen-pulsed, B7-DC XAb-activated DC, the recovered T cells were CFSE-high, FoxP3 negative in phenotype. Because no significant proliferation was observed in this group, the possible outgrowth by contaminating effector/non-Tregs cells as an explanation for the change in phenotype of the T cell population can be ruled out. Importantly, this finding also indicates that down regulation of FoxP3 in Tregs does not require cell division, in spite of a strict requirement for antigen recognition.

The numbers and percentages of positive FoxP3 cells in these cultures was consistent with the view that the changes induced were also not the result of cell death. Thus, the percentage of Tregs in absence of antigen in presence of control antibody treated DC or B7-DC XAb treated DC were 79% and 78% respectively. The percentage of Tregs recovered with antigen-pulsed, isotype-control antibody-treated DC or B7-DC XAb treated were 85% and 1.7% respectively. The same number of T cells were recovered from each culture.

### Example 20

### IL-6 is Required for Down Modulating the Tregs Phenotype In Vitro and In Vivo

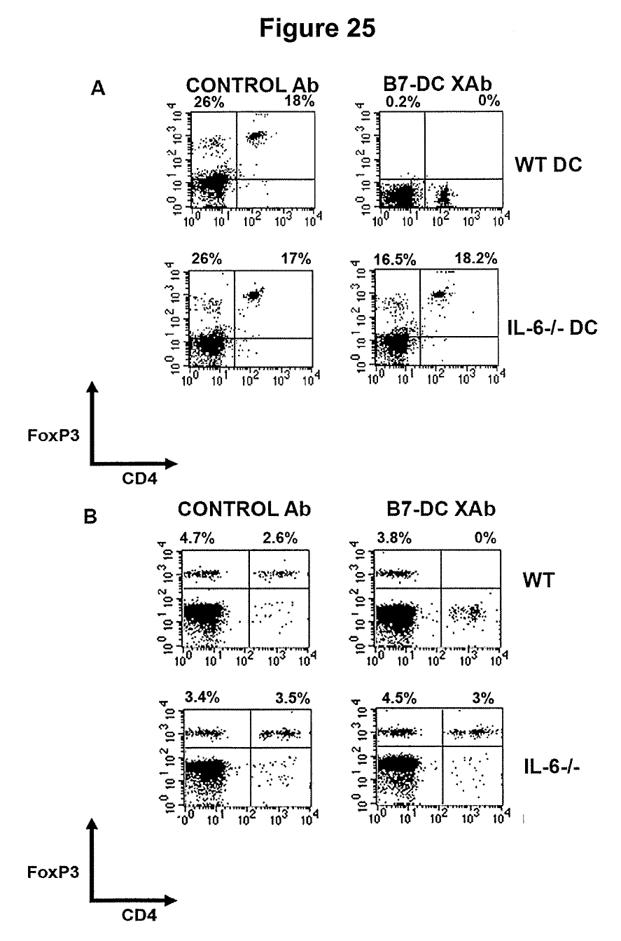

IL-6 plays a dominant role in preventing newly activated T cells from differentiating into Tregs, while favoring the development of the Th17 phenotype. The importance of IL-6 expression in modulating the phenotype of established Tregs is not well understood, however. The inventors have previously shown that DC stimulated with B7-DC XAb secrete IL-6. Therefore, the importance of IL-6 in mediating suppression of FoxP3 expression in OVA specific OT-II Tregs by B7-DC XAb-activated IL-6 deficient DC was evaluated. Cultured B7-DC XAb-activated, antigen-pulsed DC from IL-6 deficient mice did not cause down regulation of FoxP3 expression by OT-II CD4+CD25+ T cells following co-culture of the two cell populations in vitro (FIG. 25A). When OT-II Tregs were adoptively transferred into wild-type mice along with B7-DC XAb-activated, antigen-pulsed WT DC or DC from IL-6 deficient mice, FoxP3 was down regulated by B7-DC XAb-activated WT DC, but not by activated DC from IL-6 deficient mice (FIG. 25B).

### Example 21

### B7-DC XAb Breaks Tolerance in an IL-6 Dependent Manner

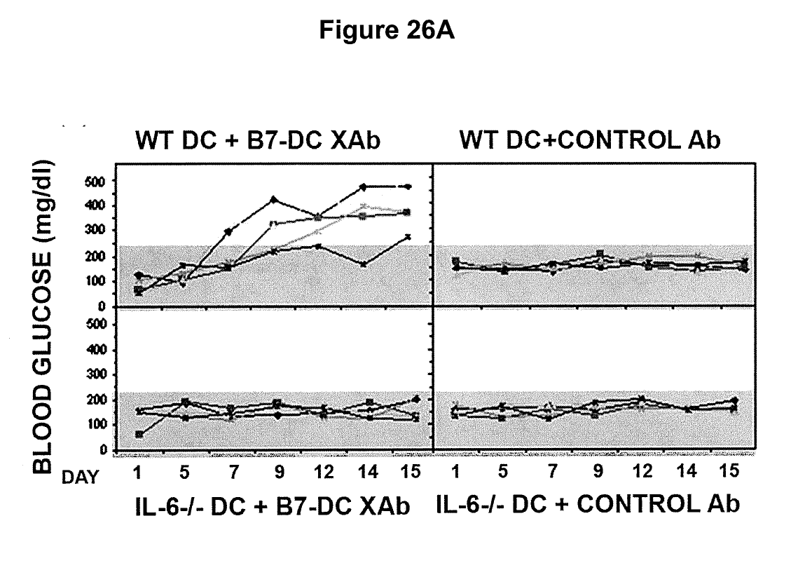

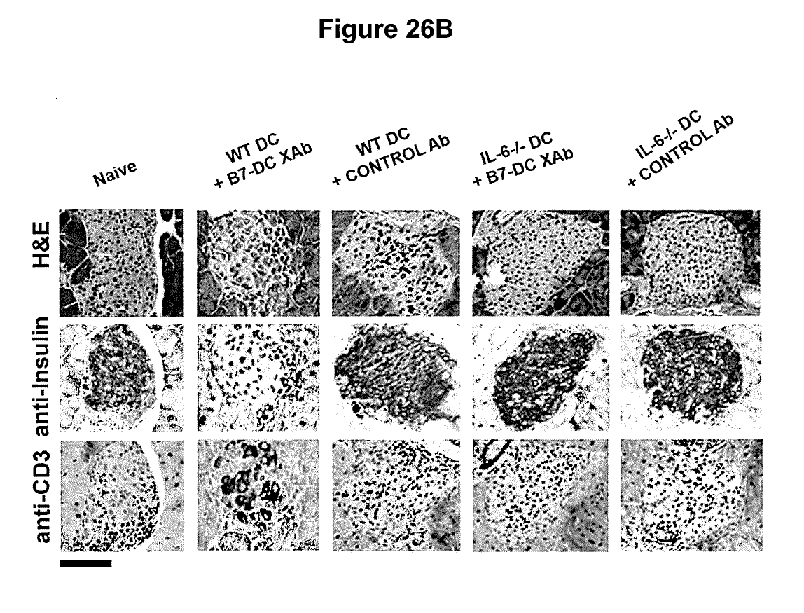

Experiments were conducted to address the functional relevance of Treg dysregulation in mice. The inventors have previously demonstrated that administration of B7-DC XAb rapidly induces CTL against soluble and cellular antigens. RIP-OVA mice, expressing chicken ovalbumin (OVA) under the rat insulin promoter, treat the neo-antigen OVA as a self protein, become tolerant to the antigen and develop normal pancreas morphology without evidence of inflammation. To evaluate whether B7-DC XAb-activated DC could modulate tolerance to this endogenous neo-antigen, RIP-OVA mice were injected with OVA-pulsed DC that had been pulsed with ovalbumin and activated with isotype control antibody or B7-DC XAb. The DC were derived from wild-type or IL-6 deficient DC. Activated OVA-specific OT-I effector T cells were administered intraperitoneally to RIP-OVA mice as a positive control. All groups of animals were periodically monitored for their blood glucose levels. As shown in FIG. 26A, mice that received DC pulsed with antigen and control antibody retained normal glucose levels, whereas mice that received DC pulsed with antigen and treated with B7-DC XAb became diabetic (glucose levels greater than 250 mg/dL) within ten days. RIP-OVA mice that received OT-I T cells and ovalbumin in CFA also became diabetic in that time frame. Importantly, administration of IL-6 deficient B7-DC XAb-treated and antigen-pulsed DC failed to induce diabetes in any of the mice.

Blood glucose phenotypes correlated with the integrity of the pancreas in various treatment groups. Islets were absent in the diabetic mice (FIG. 26B). Furthermore, immunohistochemical staining of the pancreas for T cells and insulin (FIG. 26C) revealed extensive infiltration of T cells and the absence of insulin in diabetic mice. Mice that were non-diabetic had intact islets with no evidence of T cell infiltration. It can be concluded from these studies that IL-6 expression by the adoptively transferred DC was required for the induction of diabetes in the RIP-OVA mice. The requirement for IL-6 is consistent with the interpretation that the antigen-pulsed, B7-DC XAb-activated DC induce disease, in part, by down regulating Treg function.

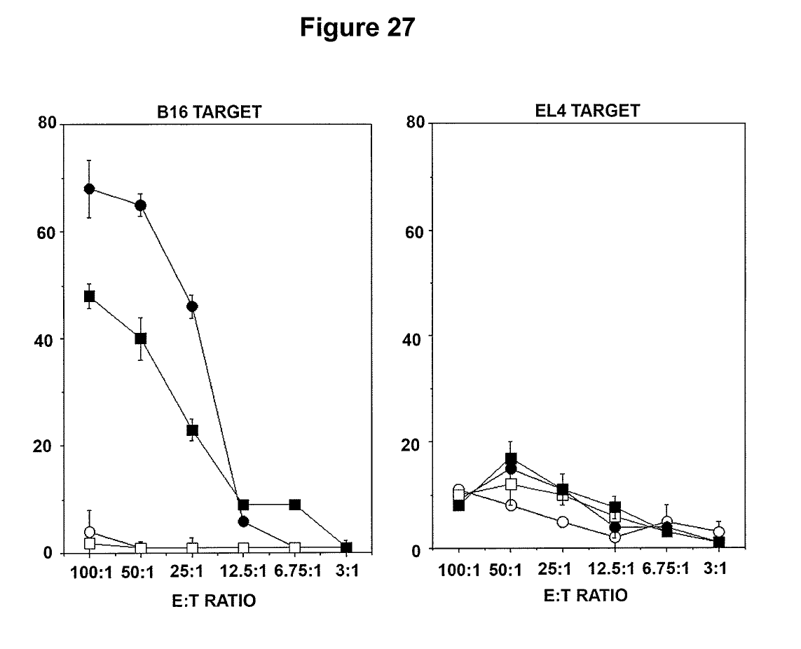

Anti-tumor immunity induced by B7-DC XAb is associated with a rapid induction of tumor-specific CTL. The functional importance of these activated effectors can be directly demonstrated by blocking their effector function or by deleting tumor-specific T cell clones by pretreating animals with soluble antigen in the absence of adjuvant. While treatment of C57BL/6 mice with B7-DC XAb protected the mice from otherwise lethal B16 melanoma grafts, similar treatment of IL-6 knockout mice was not protective (Table 3). Rapid activation of tumor-reactive CTL was induced in both wild type and IL-6 knockout mice (FIG. 27), a finding consistent with the hypothesis that an important step in B7-DC XAb-induced anti-tumor immunity is the antigen-specific regulation of Treg function.

TABLE 3
B7-DC XAb protection against tumor is IL-6 dependent

| Group | Tumor Burden |
|---|---|
| WT control Ab | 5/5 |
| WT B7-DC XAb | 0/5 |
| IL-6^{-/-} control Ab | 7/7 |
| IL-6^{-/-} B7-DC XAb | 7/7 |

### Example 22

### B7-DC XAb Does Not Induce Generalize Autoimmunity

Because treatment of mice with B7-DC XAb rendered otherwise tolerant animals responsive to antigens associated with self proteins in both the Her2/neu and RIP-OVA transgenic models, studies were conducted to assess whether the antibody might induce generalized autoimmunity when administered systemically to animals. Twenty male BALB/c mice, housed individually, were randomized and received either 200 ug B7-DC XAb or PBS, intravenously. The animals were scored over a two week period in a blinded manner for behavior and weight. Blood, heart, brain, spleen, kidney, liver, and lungs from each of the coded animals were examined for signs of abnormality by a certified pathologist. During the course of the two week observation period, one PBS treated mouse died. There were no significant differences in body weight, blood chemistry or cellularity, and no changes in organ morphology (including an absence of inflammation) among the surviving animals. Therefore, treatment of animals with B7-DC XAb did not induce a generalized autoimmunity in normal mice.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for modulating a state of immune responsiveness in a mammal, said method comprising administering to said mammal an activated dendritic cell contacted by (a) a B7-DC cross-linking molecule, and (b) an antibody directed to a component of the T-cell receptor complex.

2. The method of claim 1, wherein said B7-DC cross-linking molecule is an IgM antibody.

3. The method of claim 2, wherein said IgM antibody recognizes a B7-DC epitope comprising a glycosylation site.

4. The method of claim 1, wherein said B7-DC crosslinking molecule comprises an amino acid sequence that is between 80.0% and 99.9% identical to the amino acid sequence set forth in SEQ ID NO:3 or SEQ ID NO:5.

5-7. (canceled)

8. The method of claim 1, wherein said B7-DC crosslinking molecule comprises an amino acid sequence that is between 80.0% and 99.9% identical to the amino acid sequence set forth in SEQ ID NO:3 or SEQ ID NO:5, and further comprises an amino acid sequence that is at least 80.0% identical to the amino acid sequence set forth in SEQ ID NO:4, SEQ ID NO:6, SEQ ID NO:7, SEQ ID NO:8, or SEQ ID NO:9.

9. The method of claim 1, wherein said B7-DC cross-linking molecule is B7-DC XAb.

10. The method of claim 1, wherein said antibody directed to a component of the T-cell receptor complex is an anti-CD3 antibody.

11. The method of claim 1, wherein said antibody directed to the T-cell receptor complex contacts an Fc receptor of said dendritic cell.

12. The method of claim 1, wherein said administering is intravenous.

13. A method for inhibiting tumor growth in a mammal having or at risk for having a tumor, said method comprising administering to said mammal an activated dendritic cell contacted by (a) a B7-DC cross-linking molecule and (b) an antibody directed to a component of the T-cell receptor complex.

14. The method of claim 13, wherein said B7-DC cross-linking molecule is an IgM antibody.

15. The method of claim 14, wherein said IgM antibody recognizes a B7-DC epitope comprising a glycosylation site.

16. The method of claim 13, wherein said B7-DC crosslinking molecule comprises an amino acid sequence that is between 80.0% and 99.9% identical to the amino acid sequence set forth in SEQ ID NO:3 or SEQ ID NO:5.

17-19. (canceled)

20. The method of claim 13, wherein said B7-DC crosslinking molecule comprises an amino acid sequence that is between 80.0% and 99.9% identical to the amino acid sequence set forth in SEQ ID NO:3 or SEQ ID NO:5, and further comprises an amino acid sequence that is at least 80.0% identical to the amino acid sequence set forth in SEQ ID NO:4, SEQ ID NO:6, SEQ ID NO:7, SEQ ID NO:8, or SEQ ID NO:9.

21. The method of claim 13, wherein said B7-DC cross-linking molecule is B7-DC XAb.

22-24. (canceled)

25. A composition comprising B7-DC XAb-activated dendritic cells contacted by an antibody directed to a component of the T-cell receptor complex.

26. The composition of claim 25, wherein said B7-DC cross-linking molecule is an IgM antibody.

27. (canceled)

28. The composition of claim 25, wherein said B7-DC cross-linking molecule is B7-DC XAb.

29. The composition of claim 25, wherein said antibody directed to a component of the T-cell receptor complex is an anti-CD3 antibody.

30. The composition of claim 25, wherein said antibody directed to the T-cell receptor complex contacts an Fc receptor of said dendritic cell.
